(19) United States
(12) Patent Application Publication
(10) Pub. No.: US 2020/0241011 A1
(43) Pub. Date: Jul. 30, 2020

(54) COMPOSITIONS AND METHODS RELATED TO SEX- SPECIFIC METABOLIC DRIVERS IN ALZHEIMERS DISEASE

(71) Applicants: Mattias ARNOLD, Neuherberg (DE); Gabi KASTENMUELLER, Neuherberg (DE); Kwangsik NHO, Indianapolis, IN (US); Andrew J. SAYKIN, Indianapolis, IN (US); Rui CHANG, New York, NY (US); DUKE UNIVERSITY, Durham, NC (US); HELMHOLTZ ZENTRUM MUNCHEN, Neutherberg (DE); INDIANA UNIVERSITY SCHOOL OF MEDICINE, Indianapolis, IN (US); MOUNT SINAI SCHOOL OF MEDICINE, New York, NY (US)

(72) Inventors: Rima F. Kaddurah-Daouk, Belmont, MA (US); Matthias Arnold, Neuherberg, Bavaria (DE); Gabi Kastenmueller, Neuherberg, Bavaria (DE); Kwangsik Nho, Indianapolis, IN (US); Andrew J. Saykin, Indianapolis, IN (US); Rui Chang, New York, NY (US)

(21) Appl. No.: 16/488,559

(22) PCT Filed: Feb. 24, 2018
(86) PCT No.: PCT/US2018/019601

§ 371 Date: Aug. 23, 2019

Related U.S. Application Data
(60) Provisional application No. 62/463,189, filed on Feb. 24, 2017.
(60) Provisional application No. 62/556,282, filed on Sep. 8, 2017.

(51) Int. Cl. G01N 33/68; G01N 33/92; A61B 5/055; A61B 5/00

(52) U.S. Cl. CPC G01N 33/6896; G01N 33/92; A61B 5/055; G01N 2405/04; A61B 5/0042; G01N 2800/2821; G01N 2800/52; A61B 5/4088

(57) ABSTRACT

The present disclosure relates to compositions and methods for use in the analysis of broad metabolic changes associated with neurological disorders. In particular, the present disclosure provides materials and methods relating to the use of metabolomics as a biochemical approach to identify sex-specific metabolic biomarkers of neurological disorders. Embodiments of the present disclosure include the use of sex-specific metabolic biomarkers to aid in the determination of whether a subject suffers from, or is at risk of developing, a neurological disorder, such as Alzheimer's disease (AD).

### RELATED APPLICATIONS

This application claims priority to and the benefit of U.S. Provisional Patent Application Ser. No. 62/463,189 filed Feb. 24, 2017, and U.S. Provisional Patent Application Ser. No. 62/556,282 filed Sep. 8, 2017. These applications are incorporated herein by reference in their entirety for all purposes.

### GOVERNMENT FUNDING

The subject matter of this invention was made with Government support under Federal Grant No. R01AG04617 awarded by the National Institutes on Aging (NIA). The Government has certain rights to this invention.

### TECHNICAL FIELD

Embodiments of the present disclosure relate generally to the analysis of broad metabolic changes associated with neurological disorders. In particular, the present disclosure provides materials and methods relating to the use of metabolomics as a biochemical approach to identify sex-specific metabolic biomarkers of neurological disorders. Embodiments of the present disclosure include the use of sex-specific metabolic biomarkers to aid in the determination of whether a subject suffers from, or is at risk of developing, a neurological disorder, such as Alzheimer's disease (AD).

### BACKGROUND

Alzheimer's (AD) is a progressive neurodegenerative disorder, which currently has no cure or preventive therapy, and its symptomatic therapies are only modestly effective. The failure of hundreds of trials of disease-modifying therapeutics, including several targeting amyloid-beta (Aβ), highlights our incomplete knowledge of both cause of AD and mechanisms of cognitive failure. A large number of biochemical processes are affected in disease including glucose cholesterol mitochondrial energetics and lipid metabolism. Several metabolic changes happen early and are noted in blood prior to symptoms development. Building evidence suggests that brain metabolic state is influenced by peripheral metabolic functions as well as by gut microbiome activity and environmental exposures where AD is seen as the failure of an integrated system.

Additionally, there is clear evidence that AD is more prevalent in women. For example, recent data using brain imaging, post-mortem analyses, and genetics suggest that AD affects men and women differently. In many cases, women exhibit poorer cognitive profiles as compared to men at the same stage of AD, and men have been shown to significantly outperform women in several cognitive domains, including language, episodic memory, and semantic abilities. These and other sex-specific effects of AD are poorly understood at the molecular level, and genetic analysis has provided limited insights. Thus, there is an unmet need for more gender specific diagnosing and treating of Alzheimer's disease in subjects.

### SUMMARY

The present disclosure is directed to a method of diagnosing or detecting a cognitive disorder in a subject, the method comprising: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; wherein if the subject has a level of the at least one biomarker metabolite that is lower that the level of the at least one biomarker metabolite in a control sample, the subject is diagnosed with having a cognitive disorder.

The present disclosure is directed to a method of aiding in the determination of whether to perform a head magnetic resonance imaging (MRI) procedure on a subject suspected on having a cognitive disorder, the method comprising: a) obtaining a sample from a subject, and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; c) determining that the subject has an increased risk of cortical thinning if the subject has a level of the at least one biomarker metabolite that is lower that the level of the at least one biomarker metabolite in a control sample; and d) performing a head MRI procedure on the subject that is determined to have cortical thinning.

The present disclosure is directed to a method of predicting the outcome of a subject suspected of having Alzheimer's disease, the method comprising: a) obtaining a sample from a subject; b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; wherein if the subject has a level of the at least one biomarker metabolite that is lower that the level of the at least one biomarker metabolite in a control sample, the subject is predicted to develop Alzheimer's disease, or an increased risk of Alzheimer's disease.

The present disclosure is directed to a method of diagnosing or detecting Alzheimer's disease in a subject. The method comprises: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; wherein if the subject has a level of the at least one biomarker metabolite that is lower that the level of the at least one biomarker metabolite in a control sample, the subject is diagnosed with having Alzheimer's disease.

The present disclosure is directed to a method of determining the progression of Alzheimer's disease in a subject. The method comprises: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; wherein if the subject has a level of the at least one biomarker metabolite that is lower that the level of the at least one biomarker metabolite in a control sample, the Alzheimer's disease is determined to be progressing.

The present disclosure is directed to a method of determining an increased risk of developing Alzheimer's disease in a subject. The method comprises: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; wherein if the subject has a level of the at least one biomarker metabolite that is lower that the level of the at least one biomarker metabolite in a control sample, the subject is determined to have an increased risk of developing Alzheimer's disease.

The present disclosure is directed to a method of aiding in the determination of whether to perform a head magnetic resonance imaging (MRI) procedure on a subject suspected on having Alzheimer's disease. The method comprises: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; c) determining that the subject has an increased risk of cortical thinning if the subject has a level of the at least one biomarker metabolite that is lower that the level of the at least one biomarker metabolite in a control sample; and d) performing a head MRI procedure on the subject that is determined to have cortical thinning.

The present disclosure is directed to a method of predicting the outcome of a subject suspected of having Alzheimer's disease. The method comprises: a) obtaining a sample from a subject; b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; wherein if the subject has a level of the at least one biomarker metabolite that is lower that the level of the at least one biomarker metabolite in a control sample, the subject is predicted to develop Alzheimer's disease or have an increased risk of Alzheimer's disease.

The present disclosure is directed to a method of treating a subject suspected of having Alzheimer's disease. The method comprises: a) obtaining a sample from a subject; b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof, wherein if the subject has a level of the at least one biomarker metabolite that is lower that the level of the at least one biomarker metabolite in a control sample, the subject is predicted to develop Alzheimer's disease or have an increased risk of Alzheimer's disease, and c) initiating treatment for Alzheimer's disease in the subject predicted to develop Alzheimer's disease.

### BRIEF DESCRIPTION OF THE DRAWINGS

FIG. 1 shows a metabolic predictive network for female and male in Alzheimer's disease (AD) and cognitively normal (CN).

FIG. 2 shows a metabolic predictive network for female Alzheimer's disease (AD) and cognitively normal (CN).

FIG. 3 shows a metabolic predictive network for male Alzheimer's disease (AD) and cognitively normal (CN).

FIG. 4 shows metabolic drivers in men and in women in Alzheimer Diseases.

FIG. 5 shows gender differences in metabolite levels in Alzheimer's disease Neuroimaging Initiative (ADNI) participants that are cognitively normal (CN).

FIG. 6 shows gender differences in metabolite levels in ADNI participants having Late Mild Cognitive Impairment (LCMI).

FIG. 7 shows gender differences in metabolite levels in ADNI participants with Alzheimer's disease (AD).

FIG. 8 shows a statistical model used to determine the association of branched-chain amino acid (BCAA) with imaging phenotypes men and women with Alzheimer's disease.

FIG. 9 shows low levels of valine correlate with cortical thinning in AD.

FIG. 10 shows low levels of valine correlate with cortical thinning in AD is driven by men and not women.

FIG. 11 shows low levels of isoleucine correlate with cortical thinning in AD.

FIG. 12 shows low levels of isoleucine correlate with cortical thinning in AD is driven by men and not women.

FIG. 13 shows low levels of leucine correlate with cortical thinning in AD.

FIG. 14 shows low levels of leucine correlate with cortical thinning in AD is driven mainly by men.

FIG. 15 shows Statistical Methods used in the mGWAS analysis.

FIG. 16 shows the association of isoleucine levels with ADAM9 and ADAM32.

FIG. 17 shows the association of leucine levels with CDH22 and SLC35C2.

FIG. 18 shows the association of valine levels with KLF15 and CCDC37.

FIG. 19 shows the association of valine levels with CDYL.

FIG. 20 shows the sub-networks of the target network linking key targets and metabolites.

### DETAILED DESCRIPTION

The present disclosure is based, in part, on the discovery that there are different metabolic pathways that contribute to Alzheimer's disease (AD) pathology in males and females. Hence, therapeutic targets could prove more successful if they are gender-specific. The inventors have generated vast biochemical data from Alzheimer study ADNI on close to 800 subjects and have used network approaches (Causative and partial networks) to reveal that branched chain amino acids (BCAA), such as leucine, valine, and isoleucine, and other amines, such as 2 Amino adipic acid, glutamine, tyrosine, and tryptophan, are drivers of disease in men. Low levels of valine, leucine, and isoleucine correlated with imaging and cognitive changes in men with AD. Lipids within the PC and LPC classes contributed to disease low levels correlate with cognitive decline. The inventors further discovered that there was an association between the BCAA and genes that are involved directly or indirectly with cognitive diseases, such as AD. For example, low levels of valine was associated with CDYL which is known to regulate REST, which is associated with cognitive disorders, such as AD, in men. This approach for therapy can be gender specific.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Cognitive disorders” as used herein refers to a category of mental health disorders that primarily affect cognitive abilities including learning, memory, perception, and problem solving. Cognitive disorders, also known as neurocognitive disorders, include delirium and mild and major neurocognitive disorder (previously known as dementia). Cognitive disorders are defined by deficits in cognitive ability that are acquired (as opposed to developmental), typically represent decline, and may have an underlying brain pathology. The DSM-5 defines six key domains of cognitive function: executive function, learning and memory, perceptual-motor function, language, complex attention, and social cognition. Various medical conditions can affect mental functions such as memory, thinking, and the ability to reason. Cognitive disorders can include Alzheimer's disease, frontotemporal degeneration, Huntington's disease, Lewy body disease, traumatic brain injury (TBI), Parkinson's disease, prion disease, and dementia/neurocognitive issues due to HIV infection.

“Sample,” “test sample,” “specimen,” “sample from a subject,” and “patient sample” as used herein may be used interchangeable and may be a sample of blood, such as whole blood, tissue, urine, serum, plasma, amniotic fluid, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal and a human. In some embodiments, the subject may be a human or a non-human. The subject or patient may be undergoing forms of treatment. “Mammal” as used herein refers to any member of the class Mammalia, including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The term does not denote a particular age or sex. Thus, adult and newborn subjects, as well as fetuses, whether male or female, are intended to be included within the scope of this term.

“Treat,” “treating” or “treatment” are each used interchangeably herein to describe reversing, alleviating, or inhibiting the progress of a disease, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. SEX-SPECIFIC METABOLIC DRIVERS IN ALZHEIMER'S DISEASE

Embodiments of the present disclosure relate generally to the analysis and identification of gender specific global metabolic changes in cognitive disorders, such as Alzheimer's disease (AD). More particularly, the present disclosure provides materials and methods relating to the use of metabolomics as a biochemical approach to identify gender specific metabolic changes in cognitive disorder patients, such as AD patients, as well as identify gender specific targets for treatment.

The present disclosure, baseline serum samples were profiled from the Alzheimer's Disease Neuroimaging Initiative-1 (ADNI-1) cohort where vast data exist on each patient including cognitive decline and imaging changes over many years, information on CSF markers, genetics, and other-omics data. The relationship of metabolites with longitudinal cognitive and imaging changes helped us define gender specific metabolic signatures correlated with disease progression. Key associations were also present in multiple independent cohorts. The systems approach described in the present disclosure facilitated the elucidation of metabolic changes along different stages during the progression of AD, and led to the identification of valuable gender specific biomarkers that can inform and accelerate clinical trials.

The present disclosure provides the biochemical knowledge about disease mechanisms that can be used as a roadmap for novel drug discovery and establishment of blood-based biomarkers. Eight complementary, targeted and non-targeted, metabolomics platforms are currently in the process of generating data on ADNI participants to define the metabolic trajectory of disease connecting central and gender specific metabolic failures in a pathway and network context. The present disclosure expands on biochemical coverage to better understand disease pathogenesis by using complementary data unique to ADNI-1. The unique opportunity of having longitudinal cognitive and imaging data on each subject for close to a decade enables identification of gender specific biomarkers that are disease related.

Accordingly, the present disclosure represents the first use of a targeted, highly validated metabolomics platform with the analysis guided by CSF markers and imaging data. Using 732 base-line serum samples from the ADNI-1 cohort, relationships between metabolomics data and cross-sectional clinical, CSF, and MRI measures were systematically evaluated, as well as their association with longitudinal cognitive and brain volume changes. Multiple comparisons and covariate-adjusted analyses, that included relevant medications, identified sets of metabolites that became altered at specific disease stages (preclinical AD with biomarker-defined AD pathology vs. symptomatic stages). Using partial correlation networks, the results of the present disclosure integrates data on the metabolic effects on AD pathogenesis, linking central and gender specific metabolism in a way that consistently addresses biochemical trajectories of disease with this established temporal sequence of pathophysiological stages of AD.

The present disclosure provides the material and methods pertaining to the use of metabolomics and network approaches to identify lipid metabolic changes related to early stages of AD, as well as later changes related to mitochondrial energetics and energy utilization. The lipid changes identified herein reflect alterations in membrane structure and function early in the disease process and suggest a change in lipid rafts, which in turn, cause alterations in AB processing. Over time, the changes in lipid membranes, particularly mitochondrial membranes, may result in increased lipid oxidation, loss of membrane potential, and changes in membrane transport. In some cases, lipid membrane changes might involve disruptions in BCAA as an energy source, production of acylcarnitines, and altered energy substrate utilization.

Amino acids are the monomeric building blocks of proteins, which in turn comprise a wide range of biological compounds, including enzymes, antibodies, hormones, transport molecules for ions and small molecules, collagen, and muscle tissues. Amino acids are considered hydrophobic or hydrophilic, based upon their solubility in water, and, more particularly, on the polarities of their side chains. Amino acids having polar side chains are hydrophilic, while amino acids having nonpolar side chains are hydrophobic. The solubilities of amino acids, impart, determines the structures of proteins. Hydrophilic amino acids tend to make up the surfaces of proteins while hydrophobic amino acids tend to make up the water-insoluble interior portions of proteins. Of the common 20 amino acids, nine are considered essential in humans, as the body cannot synthesize them. Rather, these nine amino acids are obtained through an individual's diet. A deficiency of one or more amino acids can cause various imbalances and can lead to the development of a disease condition(s). Additionally, as described herein, the presence or absence of one or more amino acids can indicate metabolic imbalances reflective of disease conditions, such as Alzheimer's disease. Branched chain amino acids (BCAAs), which include valine, leucine, and isoleucine, are among a subgroup of amino acids that can be predictive of the development of Alzheimer's disease. As such, BCAAs can be used to treat such conditions as they have been shown to function not only as protein building blocks, but also as inducers of signal transduction pathways that modulate translation initiation.

### 3. METHOD OF DIAGNOSING OR DETECTING A COGNITIVE DISORDER, SUCH AS ALZHEIMER'S DISEASE, IN A SUBJECT

In some embodiments, the present disclosure provides method of diagnosing or detecting a cognitive disorder in a subject. The method comprises: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof. If the subject has a level of the at least one biomarker metabolite that is lower that the level of the at least one biomarker metabolite in a control sample, the subject is diagnosed with having a cognitive disorder. In some embodiment, the cognitive disorder can be Alzheimer's disease, frontotemporal degeneration, Huntington's disease, Lewy body disease, traumatic brain injury (TBI), Parkinson's disease, prion disease, and dementia/neurocognitive issues due to HIV infection. In some embodiment, the cognitive disorder can be Alzheimer's disease.

In some embodiments, the present disclosure also provides method of diagnosing or detecting Alzheimer's disease in a subject. The method comprises: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof. If the subject has a level of the at least one biomarker metabolite that is lower that the level of the at least one biomarker metabolite in a control sample, the subject is diagnosed with having Alzheimer's disease.

In some embodiments, the subject is male and the at least one biomarker metabolite is a branched-chain amino acid biomarker metabolite, a glutamate biomarker metabolite, a tryptophan biomarker metabolite, a tyrosine biomarker metabolite, or a 2 amino adipic acid biomarker metabolite. In some embodiments, the branched-chain amino acid biomarker metabolite is valine, leucine, or isoleucine.

In some embodiments, the subject is female and the at least one biomarker metabolite is a phosphatidylcholine biomarker metabolite, a lysophosphatidylcholine (lysoPC) biomarker metabolite. In some embodiments, the phosphatidylcholine biomarker metabolite is at least one of Phosphatidylcholine diacyl C36:5 (PC as C36:5), Phosphatidylcholine diacyl C36:6 (PC aa C36:6), Phosphatidylcholine acyl-alkyl C38:0 (PC ae C38:0), Phosphatidylcholine diacyl C38:6 (PC aa C38:6), Phosphatidylcholine acyl-alkyl C40:1 (PC ae C40:1), Phosphatidylcholine diacyl C40:6 (PC aa C40:6), or combinations thereof. In some embodiments, the lysoPC biomarker metabolite is at least one of lysophosphatidylcholine a C18:2 (lysoPC a C18:2), lysophosphatidylcholine a C18:1 (lysoPC a C18:1), or combinations thereof.

In some embodiments, the method further comprises initiating treatment for Alzheimer's disease in the subject that has a level of the at least one biomarker metabolite that is lower that the level of the at least one biomarker metabolite in a control sample. In some embodiments, if the subject is male, the treatment comprises administering a composition that modulates CDYL, KLF15, CDH22, SLC35C2, ADAM32, and/or ADAM9 activity. In some embodiments, the composition comprises the branched-chain amino acid biomarker metabolite or a drug that modulates the levels of the branched-chain amino acid biomarker metabolite. In some embodiments, the treatment comprises administering a composition that modulates CDYL and the composition comprises valine or a drug that modulates the levels of valine. In some embodiments, the composition comprises a branched-chain amino acid, such as valine, isoleucine, and/or leucine.

In some embodiments, the method further comprises initiating treatment for Alzheimer's disease in the subject diagnosed with Alzheimer's disease. In some embodiments, if the subject is male and diagnosed with Alzheimer's disease or determined to have an increased risk of developing Alzheimer's disease, the treatment comprises administering a drug that modulates KLF15, CDYL, CDH22, SLC35C2, ADAM32, and/or ADAM9 activity. In some embodiments, if the at least one metabolic biomarker is valine, the drug modulates the activity of CDYL, KLF15 and/or CCDC37. In some embodiments, if the at least one metabolic biomarker is leucine, the drug modulates the activity of CDH22 and/or SLC35C2. In some embodiments, if the at least one metabolic biomarker is isoleucine, the drug modulates the activity of ADAM9 and/or ADAM32. In some embodiments, treatment comprises a branched-chain amino acid, such as valine, isoleucine, and/or leucine.

### 4. METHOD OF DETERMINING THE PROGRESSION OF A COGNITIVE DISORDER, SUCH AS ALZHEIMER'S DISEASE, IN A SUBJECT

In some embodiments, the present disclosure provides method of determining the progression of a cognitive disorder in a subject. The method comprises: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof. If the subject has a level of the at least one biomarker metabolite that is lower that the level of the at least one biomarker metabolite in a control sample, the cognitive disorder is determined to be progressing. In some embodiment, the cognitive disorder can be Alzheimer's disease, frontotemporal degeneration, Huntington's disease, Lewy body disease, traumatic brain injury (TBI), Parkinson's disease, prion disease, and dementia/neurocognitive issues due to HIV infection. In some embodiment, the cognitive disorder can be Alzheimer's disease.

In some embodiments, the present disclosure also provides method of determining the progression of Alzheimer's disease in a subject. The method comprises: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof. If the subject has a level of the at least one biomarker metabolite that is lower that the level of the at least one biomarker metabolite in a control sample, the Alzheimer's disease is determined to be progressing.

In some embodiments, the subject is male and the at least one biomarker metabolite is a branched-chain amino acid biomarker metabolite, a glutamate biomarker metabolite, a tryptophan biomarker metabolite, a tyrosine biomarker metabolite, or a 2 amino adipic acid biomarker metabolite. In some embodiments, the branched-chain amino acid biomarker metabolite is valine, leucine, or isoleucine.

In some embodiments, the subject is female and the at least one biomarker metabolite is a phosphatidylcholine biomarker metabolite, a lysophosphatidylcholine (lysoPC) biomarker metabolite. In some embodiments, the phosphatidylcholine biomarker metabolite is at least one of Phosphatidylcholine diacyl C36:5 (PC as C36:5), Phosphatidylcholine diacyl C36:6 (PC as C36:6), Phosphatidylcholine acyl-alkyl C38:0 (PC ae C38:0), Phosphatidylcholine diacyl C38:6 (PC aa C38:6), Phosphatidylcholine acyl-alkyl C40:1 (PC ae C40:1), Phosphatidylcholine diacyl C40:6 (PC as C40:6), or combinations thereof. In some embodiments, the lysoPC biomarker metabolite is at least one of lysophosphatidylcholine a C18:2 (lysoPC a C18:2), lysophosphatidylcholine a C18:1 (lysoPC a C18:1), or combinations thereof.

In some embodiments, the method further comprises initiating treatment for Alzheimer's disease in the subject that has a level of the at least one biomarker metabolite that is lower that the level of the at least one biomarker metabolite in a control sample. In some embodiments, if the subject is male, the treatment comprises administering a composition that modulates CDYL, KLF15, CDH22, SLC35C2, ADAM32, and/or ADAM9 activity. In some embodiments, the composition comprises the branched-chain amino acid biomarker metabolite or a drug that modulates the levels of the branched-chain amino acid biomarker metabolite. In some embodiments, the treatment comprises administering a composition that modulates CDYL and the composition comprises valine or a drug that modulates the levels of valine. In some embodiments, the composition comprises a branched-chain amino acid, such as valine, isoleucine, and/or leucine.

In some embodiments, the method further comprises initiating treatment for Alzheimer's disease in the subject whose Alzheimer's disease is determined to be progressing. In some embodiments, if the subject is male and the Alzheimer's disease is determined to be progressing, the treatment comprises administering a drug that modulates KLF15, CDYL, CDH22, SLC35C2, ADAM32, and/or ADAM9 activity. In some embodiments, if the at least one metabolic biomarker is valine, the drug modulates the activity of CDYL, KLF15 and/or CCDC37. In some embodiments, if the at least one metabolic biomarker is leucine, the drug modulates the activity of CDH22 and/or SLC35C2. In some embodiments, if the at least one metabolic biomarker is isoleucine, the drug modulates the activity of ADAM9 and/or ADAM32. In some embodiments, treatment comprises a branched-chain amino acid, such as valine, isoleucine, and/or leucine.

### 5. METHOD OF DETERMINING AN INCREASED RISK OF DEVELOPING A COGNITIVE DISORDER, SUCH AS ALZHEIMER'S DISEASE, IN A SUBJECT

In some embodiments, the present disclosure provides method of determining an increased risk of developing a cognitive disorder in a subject. The method comprises: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof. If the subject has a level of the at least one biomarker metabolite that is lower that the level of the at least one biomarker metabolite in a control sample, the subject is determined to have an increased risk of developing a cognitive disorder. In some embodiment, the cognitive disorder can be Alzheimer's disease, frontotemporal degeneration, Huntington's disease, Lewy body disease, traumatic brain injury (TBI), Parkinson's disease, prion disease, and dementia/neurocognitive issues due to HIV infection. In some embodiment, the cognitive disorder can be Alzheimer's disease.

In some embodiments, the present disclosure also provides method of determining an increased risk of developing Alzheimer's disease in a subject. The method comprises: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof. If the subject has a level of the at least one biomarker metabolite that is lower that the level of the at least one biomarker metabolite in a control sample, the subject is determined to have an increased risk of developing Alzheimer's disease.

In some embodiments, the subject is male and the at least one biomarker metabolite is a branched-chain amino acid biomarker metabolite, a glutamate biomarker metabolite, a tryptophan biomarker metabolite, a tyrosine biomarker metabolite, or a 2 amino adipic acid biomarker metabolite. In some embodiments, the branched-chain amino acid biomarker metabolite is valine, leucine, or isoleucine.

In some embodiments, the subject is female and the at least one biomarker metabolite is a phosphatidylcholine biomarker metabolite, a lysophosphatidylcholine (lysoPC) biomarker metabolite. In some embodiments, the phosphatidylcholine biomarker metabolite is at least one of Phosphatidylcholine diacyl C36:5 (PC aa C36:5), Phosphatidylcholine diacyl C36:6 (PC as C36:6), Phosphatidylcholine acyl-alkyl C38:0 (PC ae C38:0), Phosphatidylcholine diacyl C38:6 (PC as C38:6), Phosphatidylcholine acyl-alkyl C40:1 (PC ae C40:1), Phosphatidylcholine diacyl C40:6 (PC as C40:6), or combinations thereof. In some embodiments, the lysoPC biomarker metabolite is at least one of lysophosphatidylcholine a C18:2 (lysoPC a C18:2), lysophosphatidylcholine a C18:1 (lysoPC a C18:1), or combinations thereof.

In some embodiments, the method further comprises initiating treatment for Alzheimer's disease in the subject that has a level of the at least one biomarker metabolite that is lower that the level of the at least one biomarker metabolite in a control sample. In some embodiments, if the subject is male, the treatment comprises administering a composition that modulates CDYL, KLF15, CDH22, SLC35C2, ADAM32, and/or ADAM9 activity. In some embodiments, the composition comprises the branched-chain amino acid biomarker metabolite or a drug that modulates the levels of the branched-chain amino acid biomarker metabolite. In some embodiments, the treatment comprises administering a composition that modulates CDYL and the composition comprises valine or a drug that modulates the levels of valine. In some embodiments, the composition comprises a branched-chain amino acid, such as valine, isoleucine, and/or leucine.

In some embodiments, the method further comprises initiating treatment for Alzheimer's disease in the subject determined to have an increased risk of developing Alzheimer's disease. In some embodiments, if the subject is male and determined to have an increased risk of developing Alzheimer's disease, the treatment comprises administering a drug that modulates KLF15, CDYL, CDH22, SLC35C2, ADAM32, and/or ADAM9 activity. In some embodiments, if the at least one metabolic biomarker is valine, the drug modulates the activity of CDYL, KLF15 and/or CCDC37. In some embodiments, if the at least one metabolic biomarker is leucine, the drug modulates the activity of CDH22 and/or SLC35C2. In some embodiments, if the at least one metabolic biomarker is isoleucine, the drug modulates the activity of ADAM9 and/or ADAM32. In some embodiments, treatment comprises a branched-chain amino acid, such as valine, isoleucine, and/or leucine.

### 6. METHOD OF AIDING IN THE DETERMINATION OF WHETHER TO PERFORM A HEAD MAGNETIC RESONANCE IMAGING (MRI) PROCEDURE ON A SUBJECT SUSPECTED ON HAVING A COGNITIVE DISORDER, SUCH AS ALZHEIMER'S DISEASE

In some embodiments, the present disclosure provides method of aiding in the determination of whether to perform a head magnetic resonance imaging (MRI) procedure on a subject suspected on having a cognitive disorder. The method comprises: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; c) determining that the subject has an increased risk of cortical thinning if the subject has a level of the at least one biomarker metabolite that is lower that the level of the at least one biomarker metabolite in a control sample; and d) performing a head MRI procedure on the subject that is determined to have cortical thinning. In some embodiment, the cognitive disorder can be Alzheimer's disease, frontotemporal degeneration, Huntington's disease, Lewy body disease, traumatic brain injury (TBI), Parkinson's disease, prion disease, and dementia/neurocognitive issues due to HIV infection. In some embodiment, the cognitive disorder can be Alzheimer's disease.

In some embodiments, the present disclosure also provides method of aiding in the determination of whether to perform a head magnetic resonance imaging (MRI) procedure on a subject suspected on having Alzheimer's disease. The method comprises: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof, c) determining that the subject has an increased risk of cortical thinning if the subject has a level of the at least one biomarker metabolite that is lower that the level of the at least one biomarker metabolite in a control sample; and d) performing a head MRI procedure on the subject that is determined to have cortical thinning.

In some embodiments, the subject is male and the at least one biomarker metabolite is a branched-chain amino acid biomarker metabolite, a glutamate biomarker metabolite, a tryptophan biomarker metabolite, a tyrosine biomarker metabolite, or a 2 amino adipic acid biomarker metabolite. In some embodiments, the branched-chain amino acid biomarker metabolite is valine, leucine, or isoleucine.

In some embodiments, the subject is female and the at least one biomarker metabolite is a phosphatidylcholine biomarker metabolite, a lysophosphatidylcholine (lysoPC) biomarker metabolite. In some embodiments, the phosphatidylcholine biomarker metabolite is at least one of Phosphatidylcholine diacyl C36:5 (PC aa C36:5), Phosphatidylcholine diacyl C36:6 (PC aa C36:6), Phosphatidylcholine acyl-alkyl C38:0 (PC ae C38:0), Phosphatidylcholine diacyl C38:6 (PC as C38:6), Phosphatidylcholine acyl-alkyl C40:1 (PC ae C40:1), Phosphatidylcholine diacyl C40:6 (PC aa C40:6), or combinations thereof. In some embodiments, the lysoPC biomarker metabolite is at least one of lysophosphatidylcholine a C18:2 (lysoPC a C18:2), lysophosphatidylcholine a C18:1 (lysoPC a C18:1), or combinations thereof.

In some embodiments, the method further comprises initiating treatment for Alzheimer's disease in the subject that has a level of the at least one biomarker metabolite that is lower that the level of the at least one biomarker metabolite in a control sample. In some embodiments, if the subject is male, the treatment comprises administering a composition that modulates CDYL, KLF15, CDH22, SLC35C2, ADAM32, and/or ADAM9 activity. In some embodiments, the composition comprises the branched-chain amino acid biomarker metabolite or a drug that modulates the levels of the branched-chain amino acid biomarker metabolite. In some embodiments, the treatment comprises administering a composition that modulates CDYL and the composition comprises valine or a drug that modulates the levels of valine. In some embodiments, the composition comprises a branched-chain amino acid, such as valine, isoleucine, and/or leucine.

In some embodiments, the method further comprises initiating treatment for Alzheimer's disease in the subject determined to have an increased risk of cortical thinning. In some embodiments, if the subject is male and determined to have an increased risk of cortical thinning, the treatment comprises administering a drug that modulates KLF15, CDYL, CDH22, SLC35C2, ADAM32, and/or ADAM9 activity. In some embodiments, if the at least one metabolic biomarker is valine, the drug modulates the activity of CDYL, KLF15 and/or CCDC37. In some embodiments, if the at least one metabolic biomarker is leucine, the drug modulates the activity of CDH22 and/or SLC35C2. In some embodiments, if the at least one metabolic biomarker is isoleucine, the drug modulates the activity of ADAM9 and/or ADAM32. In some embodiments, treatment comprises a branched-chain amino acid, such as valine, isoleucine, and/or leucine.

### 7. METHODS OF PREDICTING THE OUTCOME OF A SUBJECT SUSPECTED OF HAVING A COGNITIVE DISORDER, SUCH AS ALZHEIMER'S DISEASE

In some embodiments, the present disclosure provides method of predicting the outcome of a subject suspected of having a cognitive disorder. The method comprises: a) obtaining a sample from a subject; b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; wherein if the subject has a level of the at least one biomarker metabolite that is lower that the level of the at least one biomarker metabolite in a control sample, the subject is predicted to develop a cognitive disorder or have an increased risk of a cognitive disorder. In some embodiment, the cognitive disorder can be Alzheimer's disease, frontotemporal degeneration, Huntington's disease, Lewy body disease, traumatic brain injury (TBI), Parkinson's disease, prion disease, and dementia/neurocognitive issues due to HIV infection. In some embodiment, the cognitive disorder can be Alzheimer's disease.

In some embodiments, the present disclosure also provides method of predicting the outcome of a subject suspected of having Alzheimer's disease. The method comprises: a) obtaining a sample from a subject; b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; wherein if the subject has a level of the at least one biomarker metabolite that is lower that the level of the at least one biomarker metabolite in a control sample, the subject is predicted to develop Alzheimer's disease or have an increased risk of Alzheimer's disease.

In some embodiments, the subject is male and the at least one biomarker metabolite is a branched-chain amino acid biomarker metabolite, a glutamate biomarker metabolite, a tryptophan biomarker metabolite, a tyrosine biomarker metabolite, or a 2 amino adipic acid biomarker metabolite. In some embodiments, the branched-chain amino acid biomarker metabolite is valine, leucine, or isoleucine.

In some embodiments, the subject is female and the at least one biomarker metabolite is a phosphatidylcholine biomarker metabolite, a lysophosphatidylcholine (lysoPC) biomarker metabolite. In some embodiments, the phosphatidylcholine biomarker metabolite is at least one of Phosphatidylcholine diacyl C36:5 (PC aa C36:5), Phosphatidylcholine diacyl C36:6 (PC aa C36:6), Phosphatidylcholine acyl-alkyl C38:0 (PC ae C38:0), Phosphatidylcholine diacyl C38:6 (PC aa C38:6), Phosphatidylcholine acyl-alkyl C40:1 (PC ae C40:1), Phosphatidylcholine diacyl C40:6 (PC aa C40:6), or combinations thereof. In some embodiments, the lysoPC biomarker metabolite is at least one of lysophosphatidylcholine a C18:2 (lysoPC a C18:2), lysophosphatidylcholine a C18:1 (lysoPC a C18:1), or combinations thereof.

In some embodiments, the method further comprises initiating treatment for Alzheimer's disease in the subject that has a level of the at least one biomarker metabolite that is lower that the level of the at least one biomarker metabolite in a control sample. In some embodiments, if the subject is male, the treatment comprises administering a composition that modulates CDYL, KLF15, CDH22, SLC35C2, ADAM32, and/or ADAM9 activity. In some embodiments, the composition comprises the branched-chain amino acid biomarker metabolite or a drug that modulates the levels of the branched-chain amino acid biomarker metabolite. In some embodiments, the treatment comprises administering a composition that modulates CDYL and the composition comprises valine or a drug that modulates the levels of valine. In some embodiments, the composition comprises a branched-chain amino acid, such as valine, isoleucine, and/or leucine.

In some embodiments, the method further comprises initiating treatment for Alzheimer's disease in the subject predicted to develop Alzheimer's disease. In some embodiments, if the subject is male and predicted to develop Alzheimer's disease, the treatment comprises administering a drug that modulates KLF15, CDYL, CDH22, SLC35C2, ADAM32, and/or ADAM9 activity. In some embodiments, if the at least one metabolic biomarker is valine, the drug modulates the activity of CDYL, KLF15 and/or CCDC37. In some embodiments, if the at least one metabolic biomarker is leucine, the drug modulates the activity of CDH22 and/or SLC35C2. In some embodiments, if the at least one metabolic biomarker is isoleucine, the drug modulates the activity of ADAM9 and/or ADAM32. In some embodiments, treatment comprises a branched-chain amino acid, such as valine, isoleucine, and/or leucine.

### 8. METHOD OF TREATING A SUBJECT SUSPECTED OF HAVING A COGNITIVE DISORDER, SUCH AS ALZHEIMER'S DISEASE

In some embodiments, the present disclosure provides method of treating a subject suspected of having a cognitive disorder. The method comprises: a) obtaining a sample from a subject; b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; wherein if the subject has a level of the at least one biomarker metabolite that is lower that the level of the at least one biomarker metabolite in a control sample, the subject is predicted to develop a cognitive disorder or have an increased risk of a cognitive disorder, and c) initiating treatment for a cognitive disorder in the subject predicted to develop a cognitive disorder. In some embodiment, the cognitive disorder can be Alzheimer's disease, frontotemporal degeneration, Huntington's disease, Lewy body disease, traumatic brain injury (TBI), Parkinson's disease, prion disease, and dementia/neurocognitive issues due to HIV infection. In some embodiment, the cognitive disorder can be Alzheimer's disease.

In some embodiments, the present disclosure also provides method of treating a subject suspected of having Alzheimer's disease. The method comprises: a) obtaining a sample from a subject; b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof, wherein if the subject has a level of the at least one biomarker metabolite that is lower that the level of the at least one biomarker metabolite in a control sample, the subject is predicted to develop Alzheimer's disease or have an increased risk of Alzheimer's disease, and c) initiating treatment for Alzheimer's disease in the subject predicted to develop Alzheimer's disease.

In some embodiments, if the subject is male and determined to have an increased risk of cortical thinning, the treatment comprises administering a drug that modulates KLF15, CDYL, CDH22, SLC35C2, ADAM32, and/or ADAM9 activity. In some embodiments, if the at least one metabolic biomarker is valine, the drug modulates the activity of CDYL, KLF15 and/or CCDC37. In some embodiments, if the at least one metabolic biomarker is leucine, the drug modulates the activity of CDH22 and/or SLC35C2. In some embodiments, if the at least one metabolic biomarker is isoleucine, the drug modulates the activity of ADAM9 and/or ADAM32. In some embodiments, treatment comprises a branched-chain amino acid, such as valine, isoleucine, and/or leucine.

In some embodiments, if the subject is male, the treatment comprises administering a composition that modulates CDYL, KLF15, CDH22, SLC35C2, ADAM32, and/or ADAM9 activity. In some embodiments, the composition comprises the branched-chain amino acid biomarker metabolite or a drug that modulates the levels of the branched-chain amino acid biomarker metabolite. In some embodiments, the treatment comprises administering a composition that modulates CDYL and the composition comprises valine or a drug that modulates the levels of valine. In some embodiments, the composition comprises a branched-chain amino acid, such as valine, isoleucine, and/or leucine.

### 9. SAMPLES

As described and used herein, “sample,” “test sample,” and “biological sample” refer to fluid sample containing or suspected of containing a biomarker metabolite. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing a biomarker metabolite may be assayed directly. In one embodiment, the source containing a biomarker metabolite is a human bodily substance (e.g., bodily fluid, blood such as whole blood, serum, plasma, urine, saliva, sweat, sputum, semen, mucus, lacrimal fluid, lymph fluid, amniotic fluid, interstitial fluid, lung lavage, cerebrospinal fluid, feces, tissue, organ, or the like). Tissues may include, but are not limited to skeletal muscle tissue, liver tissue, lung tissue, kidney tissue, myocardial tissue, brain tissue, bone marrow, cervix tissue, skin, etc. The sample may be a liquid sample or a liquid extract of a solid sample. In certain cases, the source of the sample may be an organ or tissue, such as a biopsy sample, which may be solubilized by tissue disintegration/cell lysis. In some embodiments, the sample from the subject is whole blood, serum, plasma, or cerebral spinal fluid (CSF).

In some embodiments of the present disclosure, it may be desirable to include a control sample. The control sample may be analyzed concurrently with the sample from the subject as described above. The results obtained from the subject sample can be compared to the results obtained from the control sample. Standard curves may be provided, with which assay results for the sample may be compared. Such standard curves present levels of biomarker as a function of assay units (e.g., fluorescent signal intensity, biochemical indicator). Using samples taken from multiple donors, standard curves can be provided for reference levels of a biomarker metabolite in subjects with normal cognition, for example, as well as for “at-risk” levels of the biomarker metabolite in samples obtained from donors, who may have one or more of the characteristics set forth above. In some embodiments, the control sample is taken from a subject or population of subjects with normal cognition.

In accordance with these embodiments, a method for determining the presence, amount, or concentration of a biomarker metabolite in a test sample is provided. The method comprises assaying a test sample and/or a control sample for a biomarker metabolite using an assay, for example, designed to detect the metabolite itself (e.g., detectable label) and/or using an assay that compares a signal generated by a detectable label as a direct or indirect indication of the presence, amount, or concentration of a biomarker metabolite in the test sample to a signal generated as a direct or indirect indication of the presence, amount, or concentration of a control.

### 10. INDICATORS OF ALZHEIMER'S DISEASE

In some embodiments, the method further comprises determining whether the subject has at least one independent indicator of Alzheimer's disease, wherein the at least one independent indicator of Alzheimer's disease comprises at least one of an increase in Alzheimer's Disease Assessment Scale cognitive subscale 13 (ADAS-Cog 13) score, an increase in Spatial Pattern of Abnormality for Recognition of Early Alzheimer's disease (SPARE-AD) score, an increase in brain ventricular volume, presence of Amyloid β 1-42 protein fragment (Aβ1-42), an increased total Tau (T-tau)/Aβ1-42 ratio, or combinations thereof.

### 11. COMPOSITIONS, PHARMACEUTICAL COMPOSITIONS, AND FORMULATIONS

Embodiments of the present disclosure also provide compositions, pharmaceutical compositions, and formulations that include at least one branched-chain amino acid, such as valine, leucine, isoleucine, and/or combinations thereof. The disclosed compositions, pharmaceutical compositions, and formulations can be used to treat or alleviate the symptoms of subjects that are diagnosed with or determined as having an increased risk of the cognitive disorder, such as Alzheimer's disease. The disclosed compositions, pharmaceutical compositions, and formulations can include at least one branched-chain amino acid, such as valine, leucine, isoleucine, and/or combinations thereof, in amounts that may be particularly effectively in treating male subjects and/or female subjects. In some embodiments, the compositions, pharmaceutical compositions, and formulations comprise at least one branched-chain amino acid, such as such as valine, leucine, isoleucine, and/or combinations thereof, in a particular formulation that may be more effective in treating male subjects compared to female subjects. In some embodiments, the compositions, pharmaceutical compositions, and formulations comprise at least one branched-chain amino acid, such as such as valine, leucine, isoleucine, and/or combinations thereof, in a particular formulation that may be more effective in treating female subjects compared to male subjects. In some embodiments, the compositions, pharmaceutical compositions, and formulations comprise at least one branched-chain amino acid, such as such as valine, leucine, isoleucine, and/or combinations thereof, in a particular formulation that may have the same effectiveness in treating female subjects compared to male subjects.

The compositions, pharmaceutical compositions, and formulations may include a “therapeutically effective amount” or a “prophylactically effective amount” of at least one branched-chain amino acid, such as valine, leucine, isoleucine, and/or combinations thereof. A “therapeutically effective amount” refers to an amount effective, at dosages and for periods of time necessary, to achieve the desired therapeutic result. A therapeutically effective amount of the compositions may be determined by a person skilled in the art and may vary according to factors such as the disease state, age, sex, and weight of the individual, and the ability of the compositions to elicit a desired response in the individual. A therapeutically effective amount is also one in which any toxic or detrimental effects of a branched-chain amino acid, such as valine, leucine, isoleucine, and/or combinations thereof, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Embodiments of the present disclosure also provide compositions, pharmaceutical compositions, and formulations that include a composition that modulates CDYL, KLF15, CDH22, SLC35C2, ADAM32, and/or ADAM9 activity. The disclosed compositions, pharmaceutical compositions, and formulations can be used to treat or alleviate the symptoms of subjects that are diagnosed with or determined as having an increased risk of the cognitive disorder, such as Alzheimer's disease. The disclosed compositions, pharmaceutical compositions, and formulations can include a composition that modulates CDYL, KLF15, CDH22, SLC35C2, ADAM32, and/or ADAM9 activity, in amounts that may be particularly effectively in treating male subjects and/or female subjects. In some embodiments, the compositions, pharmaceutical compositions, and formulations comprise a composition that modulates CDYL, KLF15, CDH22, SLC35C2, ADAM32, and/or ADAM9 activity, in a particular formulation that may be more effective in treating male subjects compared to female subjects. In some embodiments, the compositions, pharmaceutical compositions, and formulations comprise a composition that modulates CDYL, KLF15, CDH22, SLC35C2, ADAM32, and/or ADAM9 activity, in a particular formulation that may be more effective in treating female subjects compared to male subjects. In some embodiments, the compositions, pharmaceutical compositions, and formulations comprise a composition that modulates CDYL, KLF15, CDH22, SLC35C2, ADAM32, and/or ADAM9 activity, in a particular formulation that may have the same effectiveness in treating female subjects compared to male subjects.

The compositions, pharmaceutical compositions, and formulations may include a “therapeutically effective amount” or a “prophylactically effective amount” of a composition that modulates CDYL, KLF15, CDH22, SLC35C2, ADAM32, and/or ADAM9 activity. A “therapeutically effective amount” refers to an amount effective, at dosages and for periods of time necessary, to achieve the desired therapeutic result. A therapeutically effective amount of the compositions may be determined by a person skilled in the art and may vary according to factors such as the disease state, age, sex, and weight of the individual, and the ability of the compositions to elicit a desired response in the individual. A therapeutically effective amount is also one in which any toxic or detrimental effects of a composition that modulates CDYL, KLF15, CDH22, SLC35C2, ADAM32, and/or ADAM9 activity, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of branched-chain amino acid, such as valine, leucine, isoleucine, and/or combinations thereof, calculated to produce the desired therapeutic effect in association with the required pharmaceutical carrier. The specification for the dosage unit forms are dictated by and directly dependent on (a) the unique characteristics of at least one branched-chain amino acid, such as valine, leucine, isoleucine, and/or combinations thereof, and the particular therapeutic or prophylactic effect to be achieved, and (b) the limitations inherent in the art of compounding such composition that modulates CDYL, KLF15, CDH22, SLC35C2, ADAM32, and/or ADAM9 activity, for the treatment of sensitivity in individuals. Dosage unit form as used herein refers to physically discrete units suited as unitary dosages for the mammalian subjects to be treated; each unit containing a predetermined quantity of the composition that modulates CDYL, KLF15, CDH22, SLC35C2, ADAM32, and/or ADAM9 activity, calculated to produce the desired therapeutic effect in association with the required pharmaceutical carrier. The specification for the dosage unit forms are dictated by and directly dependent on (a) the unique characteristics of a composition that modulates CDYL, KLF15, CDH22, SLC35C2, ADAM32, and/or ADAM9 activity, and the particular therapeutic or prophylactic effect to be achieved, and (b) the limitations inherent in the art of compounding such composition that modulates CDYL, KLF15, CDH22, SLC35C2, ADAM32, and/or ADAM9 activity, for the treatment of sensitivity in individuals.

It is to be noted that dosage values may vary with the type and severity of the condition to be alleviated. Further, at least one branched-chain amino acid dose may be determined by a person skilled in the art and may vary according to factors such as the disease state, age, sex, and weight of the individual, and the ability of at least one branched-chain amino acid to elicit a desired response in the individual. Further, the dose of the composition that modulates CDYL, KLF15, CDH22, SLC35C2, ADAM32, and/or ADAM9 activity may be determined by a person skilled in the art and may vary according to factors such as the disease state, age, sex, and weight of the individual, and the ability of the composition that modulates the CDH22, SLC35C2, ADAM32, and/or ADAM9 activity to elicit a desired response in the individual. The dose is also one in which toxic or detrimental effects, if any, of at least one branched-chain amino acid are outweighed by the therapeutically beneficial effects. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The compositions, pharmaceutical compositions, and formulations may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Various delivery systems are known and can be used to administer one or more of at least one branched-chain amino acid, such as valine, leucine, isoleucine, and/or combinations thereof, and a prophylactic agent or therapeutic agent useful for preventing, managing, treating, or ameliorating the cognitive disorder, such as Alzheimer's disease, or one or more symptoms thereof, e.g., encapsulation in liposomes, microparticles, microcapsules. Various delivery systems are known and can be used to administer one or more of a composition that modulates the CDH22, SLC35C2, ADAM32, and/or ADAM9 activity, and a prophylactic agent or therapeutic agent useful for preventing, managing, treating, or ameliorating the cognitive disorder, such as Alzheimer's disease, or one or more symptoms thereof, e.g., encapsulation in liposomes, microparticles, microcapsules. Methods of administering a prophylactic or therapeutic agent of the invention include, but are not limited to, parenteral administration (e.g., intradermal, intramuscular, intraperitoneal, intravenous and subcutaneous), epidurala administration, intratumoral administration, and mucosal administration (e.g., intranasal and oral routes). In a specific embodiment, prophylactic or therapeutic agents of the invention are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

If the pharmaceutical composition is administered orally, the pharmaceutical compositions can be formulated orally in the form of tablets, capsules, cachets, gelcaps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The pharmaceutical compositions may be administered by and formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use. The methods of the invention may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The pharmaceutical compositions may be formulated as neutral or salt forms. Pharmaceutically acceptable salts include those formed with anions such as those derived from hydrochloric, phosphoric, acetic, oxalic, tartaric acid, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, compositions can be dispensed with an infusion bottle containing sterile pharmaceutical grade water or saline. Where the mode of administration is by injection, an ampoule of sterile water for injection or saline can be provided so that the ingredients may be mixed prior to administration.

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application.

In certain embodiments, at least one branched-chain amino acid, such as valine, leucine, isoleucine, and/or combinations thereof, may be orally administered, for example, with an inert diluent or an assimilable edible carrier. At least one branched-chain amino acid (and other ingredients, if desired) may also be enclosed in a hard or soft shell gelatin capsule, compressed into tablets, or incorporated directly into the subject's diet. For oral therapeutic administration, at least one branched-chain amino acid may be incorporated with excipients and used in the form of ingestible tablets, buccal tablets, troches, capsules, elixirs, suspensions, syrups, wafers, and the like. To administer a branched-chain amino acid by other than parenteral administration, it may be necessary to coat at least one branched-chain amino acid with, or co-administer at least one branched-chain amino acid with, a material to prevent its inactivation.

At least one branched-chain amino acid, such as valine, leucine, isoleucine, and/or combinations thereof, can be used alone or in combination to treat the cognitive disorder, such as Alzheimer's disease, or any other disease or condition associated with the cognitive disorder. It should further be understood that the combinations are those combinations useful for their intended purpose.

In certain embodiments, the composition that modulates CDYL, KLF15, CDH22, SLC35C2, ADAM32, and/or ADAM9 activity, may be orally administered, for example, with an inert diluent or an assimilable edible carrier. The composition that modulates CDYL, KLF15, CDH22, SLC35C2, ADAM32, and/or ADAM9 activity (and other ingredients, if desired) may also be enclosed in a hard or soft shell gelatin capsule, compressed into tablets, or incorporated directly into the subject's diet. For oral therapeutic administration, the composition that modulates CDYL, KLF15, CDH22, SLC35C2, ADAM32, and/or ADAM9 activity may be incorporated with excipients and used in the form of ingestible tablets, buccal tablets, troches, capsules, elixirs, suspensions, syrups, wafers, and the like. To administer a branched-chain amino acid by other than parenteral administration, it may be necessary to coat the composition that modulates CDYL, KLF15, CDH22, SLC35C2, ADAM32, and/or ADAM9 activity with, or co-administer the composition that modulates CDYL, KLF15, CDH22, SLC35C2, ADAM32, and/or ADAM9 activity with, a material to prevent its inactivation.

The composition that modulates CDYL, KLF15, CDH22, SLC35C2, ADAM32, and/or ADAM9 activity, can be used alone or in combination to treat the cognitive disorder, such as Alzheimer's disease, or any other disease or condition associated with the cognitive disorder. It should further be understood that the combinations are those combinations useful for their intended purpose.

### 12. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

### Methods

AbsoluteIDQ-P180 Kit Metabolite Measurements.

A targeted metabolomics platform was used to measure close to 180 metabolites that cover lipids acylcarnitines and amines. Metabolites were measured with a targeted metabolomics approach using the AbsoluteIDQ® p180 Kit (BIOCRATES Life Science AG, Innsbruck, Austria) with an ultra-performance liquid chromatography (UPLC)/MS/MS system (Acquity UPLC (Waters), TQ-S triple quadrupole MS/MS (Waters)) which provides measurements of up to 186 endogenous metabolites quantitatively (amino acids and biogenic amines) and semi-quantitatively (acylcarnitines, sphingomyelins, PCs, and lyso-glycerophosphatidylcholines (lysoPCs) across multiple classes). The AbsoluteIDQ® p180 kit was fully validated according to European Medicine Agency Guidelines on bioanalytical method validation. Additionally, plates include an automated technical validation to approve the validity of the run and provide verification of the actual performance of the applied quantitative procedure including instrumental analysis. The technical validation of each analyzed kit plate was performed using MetIDQ® software based on results obtained and defined acceptance criteria for blank, zero samples, calibration standards and curves, low/medium/high-level QC samples and measured signal intensity of internal standards over the plate. This is a highly useful platform that was used in hundreds of publications, including several studies in AD.

De-identified samples were analyzed following the manufacturer's protocol, with metabolomics laboratories blinded to diagnosis and pathological data. Serum samples from all 807 ADNI-1 participants were analyzed, but after QC, a smaller number of participants were included in the analysis. Three participants were excluded due to incomplete clinical data, 70 samples were excluded due to non-fasting status, and 2 samples were excluded during the multivariate outlier detection step, leaving 732 participants included in the final analyses. Each assay plate included two sets of replicates: (1) A set of duplicates obtained by pooling the first 72 samples in the study (QC pool duplicates) and (2) 20 blinded analytical duplicates (blinded duplicates).

P180 QC.

Metabolites with >40% of measurements below the lower limit of detection (LOD) were excluded from the analysis. Metabolite values were scaled across the different plates using the QC pool duplicates. LOD values were imputed using each metabolite's LOD/2 value. Using the blinded duplicates, metabolites were selected with a coefficient of variation <20% and an intraclass correlation coefficient >0.65. Based on the QC process, 32 of the flow injection analysis (FIA) metabolites and 14 of the UPLC metabolites were excluded from further analysis. The presence of multivariate outlier participants were checked by evaluating the first and second principal components in each platform. Two multivariate outliers were beyond 7 standard deviations and were therefore excluded. For the participants with duplicated measurements, the average values of the two measured values were used in further analyses.

Study Cohorts and Samples.

ADNI-1 baseline samples—ADNI shipped 831 samples with unique identifiers belonging to 807 subjects. These initial identifiers were different from the ADNI subject identifiers. There were duplicate aliquots from the same CSF draw for 24 subjects to help us evaluate analytical performance. Only after the final raw data was submitted to ADNI, was the information obtained to link the samples identifier to the subject RID and identify the duplicates. Data were obtained from the ADNI database in September 2015 (adni.loni.usc.edu). ADNI-1 was launched in 2004 by the National Institute on Aging (NIA), the National Institute of Biomedical Imaging and Bioengineering, the Food and Drug Administration, private pharmaceutical companies and non-profit organizations. ADNI-1 patients underwent extensive clinical and cognitive testing, including the Alzheimer's Disease Assessment Scale-Cognition (ADAS-Cog13), which was used as a measure of general cognition in this analysis. AD dementia diagnosis was established based on the NINDS-ADRDA criteria for probable AD. Mild cognitive impairment (MCI) participants did not meet these AD criteria and had largely intact functional performance, meeting predetermined criteria for amnestic MCI. Controls were cognitively normal (CN). Additional details of participant selection criteria and protocol are available at adni-info.org. The study was approved by institutional review boards of all participating institutions and written informed consent was obtained from all participants and/or authorized representatives prior to study commencement.

Predictive Network Model.

Vast biochemical data generated on 800 ADNI 1 subjects (200 CN, 400LMCI and 200 AD) were analyzed using baseline samples and complimentary metabolomics platforms. Predictive network models were built on the integration of novel causality inference (bottom-up) approach with conventional top-down Bayesian networks (BN). The integrated top-down & bottom-up approach was able to discern the otherwise undistinguishable causality structures in BN and to result in a complete causal network.

Partial Correlation Networks.

Biochemically related metabolites and propagation patterns of effects on the clinical variables were investigated from a network perspective. A Gaussian graphical model (GGM) calculation was performed using the GeneNet R package with default parameters. To illustrate effect propagation on clinical variables, we colored the resulting network. In brief, a GGM is an undirected graphical model based on partial correlation coefficients, that is, pairwise correlation coefficients conditioned against correlations with all other included variables. GGMs, contrary to correlation networks, thus can reveal the direct relations between metabolites. To account for correlations between metabolites and clinical or other potentially predictive variables, metabolite residuals were used that accounted for effects of medication and dietary supplements (as described above) and additionally included age, gender, APOE ε4 presence, and education as covariates in the GGM generation process. To obtain significant partial correlations, a significance threshold of 0.05 after Bonferroni correction was used for all possible edges in the model (0.05/10,296=4.86×10−6). For each clinical variable, the network representation of the GGM was colored using the results of our regression analyses using sign(β)*(−log 10(P)) to visualize both strength of association and direction of effect.

MRI Measures.

TI-weighted brain MRI scans at baseline were acquired using a sagittal 3D magnetization-prepared rapid gradient-echo (MP-RAGE) sequence following the ADNI MRI protocol. Two widely employed automated MRI analysis techniques were independently used to process MRI scans and extract whole brain-wide and ROI (region of interest)-based neuroimaging endophenotypes (grey matter (GM) density, volume, and cortical thickness): whole-brain voxel-based morphometry (VBM) using statistical parametric mapping 8 (SPM8) and FreeSurfer V5.1. In particular, FreeSurfer was used to extract brain-wide cortical thickness by automated segmentation and parcellation. The cortical surface was reconstructed to measure thickness at each vertex on surface. The cortical thickness was calculated by taking the Euclidean distance between the grey/white boundary and the grey/cerebrospinal fluid (CSF) boundary at each vertex on surface.

Regional Analysis of Structural MRI.

Mean values (volume, cortical thickness, grey matter density) of 11 AD-related brain regions of interest (ROIs) were used as phenotypes (hippocampus volume, middle temporal cortical thickness, inferior temporal cortical thickness, amygdale volume, superior temporal cortical thickness, inferior parietal cortical thickness, precunneus cortical thickness, hippocampus GM density, mean temporal pole cortical thickness, and cerebral cortex GM volume). A linear regression approach was performed using age at baseline, gender, years of education, the number of APOE ε4 alleles, and intracranial volume (ICV) as covariates. For a mega-analysis, a dummy variable (ADNI-1=0 and ADNI-GO-2=1) was used as additional covariate. FDR (false discovery rate)-based multiple comparison adjustment with the Benjamini-Hochberg procedure was used because phenotypes were strongly correlated each other.

Unbiased Whole Brain Imaging Analysis.

The SurfStat software package (math.mcgill.ca/keith/surfstat/) was used to perform a multivariate analysis of cortical thickness and to examine the effect of bile acid profiles on brain structural changes on vertex-by-vertex bases by applying a general linear model (GLM) approach. GLMs were developed using age at baseline, gender, years of education, the number of APOE ε4 allele, and intracranial volume (ICV) as covariates. Change rate estimates were calculated using cortical thickness for each vertex from baseline and 24-month scans for each participant. A GLM was used to assess correlations of MRI change rate estimates with bile acid profiles. Age at baseline, gender, education, the number of APOE ε4 alleles, and baseline total cerebral cortex GM volume were included as covariates. For a mega-analysis, a dummy variable (ADNI-1=0 and ADNI-GO-2=1) was used as additional covariate. In the whole brain surface-based analysis, the adjustment for multiple comparisons was performed using the random field theory correction method at a 0.05 level of significance.

### Example 2

### Results

Results from Causative Networks.

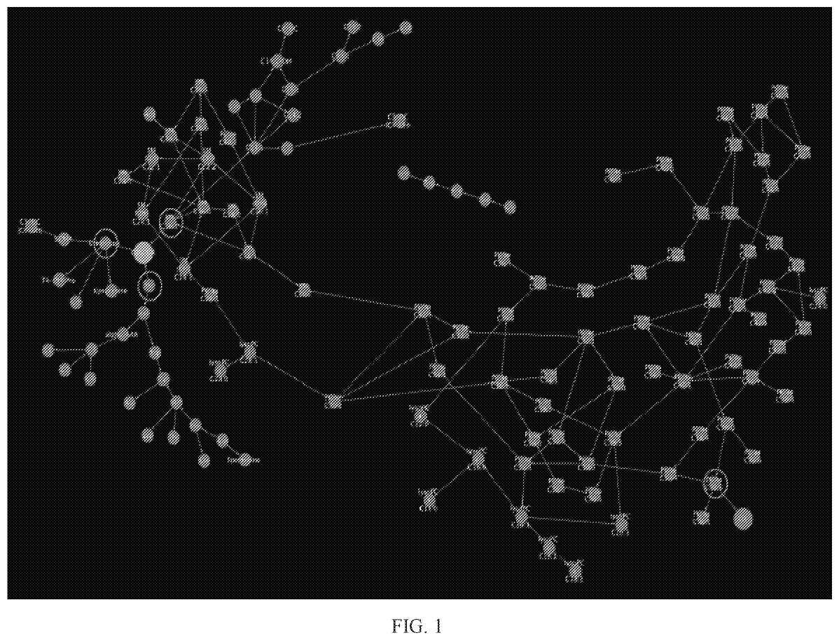

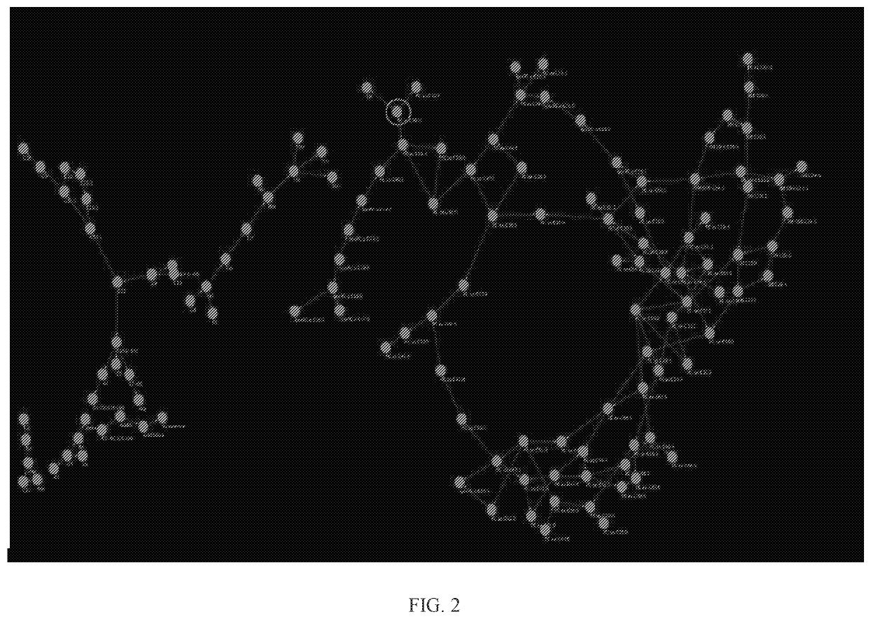

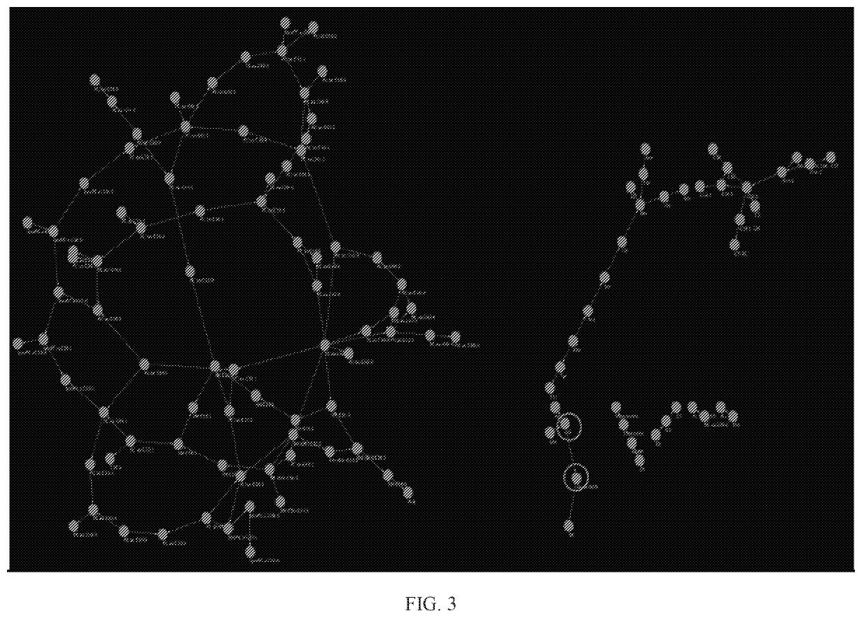

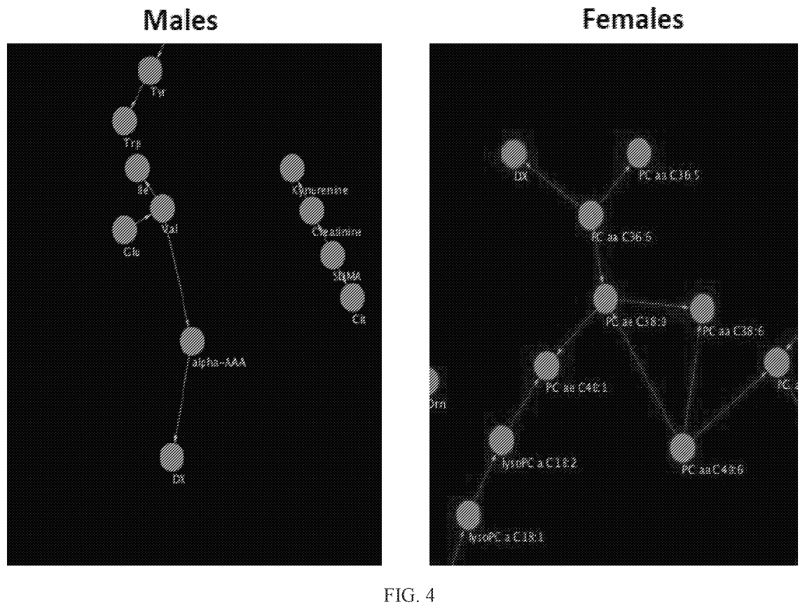

For ADNI I baseline metabolomics data, three predictive networks were built to investigate the differences in male and female metabolism in the context of AD. FIGS. 1-3 show metabolic predictive network for female and/or male in Alzheimer's disease (AD) and cognitively normal (CN). In the pooled-sex network, sphingomyelins and amines correlated with Sex and phosphatidylcholines connects to AD progression (Dx) (FIG. 1). Next, male-specific (FIG. 3) and female-specific (FIG. 2) predictive networks were built. Decreased level of several amines contributed to AD progression in males. A decreased level of Phosphatidylcholines contributes to AD progression in females. A subset of PCs formed a closely connected subnetwork in the overall network and in the female-specific network which cooperate to contribute to the AD progression. FIG. 4 shows the metabolic drivers in men and in women in Alzheimer Diseases. Drivers of disease in men were 2 Amino adipic acid, valine, glutamate, isoleucine, tryptophan tyrosine (FIG. 4). Drivers of disease in women were lipids phosphatidyl choline PC and Lyso PC.

Results from Partial Correlation Networks.

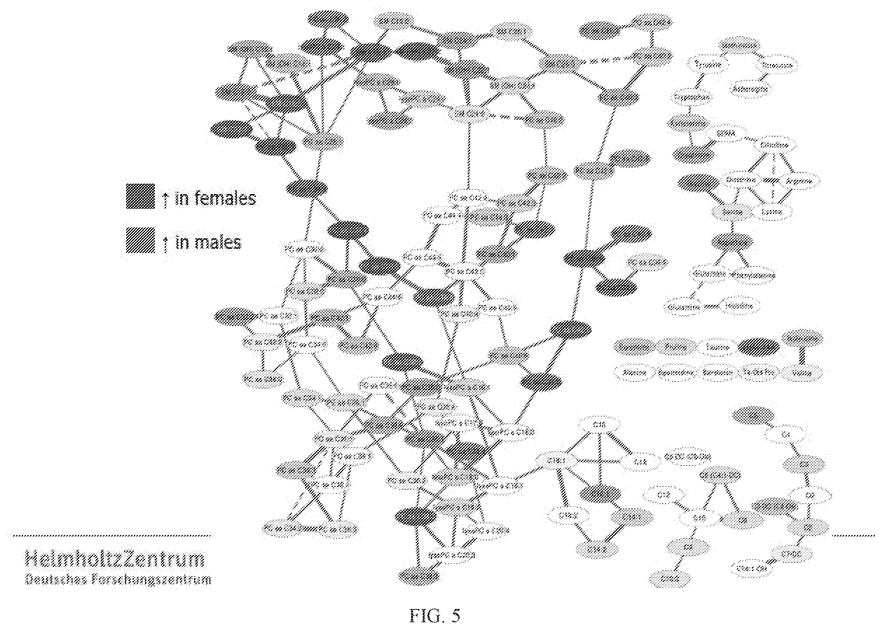

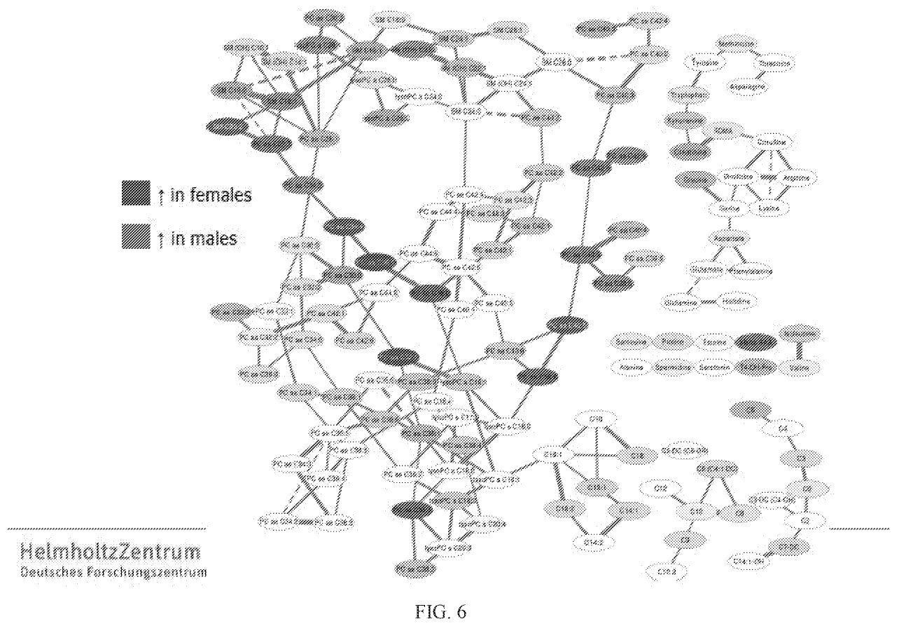

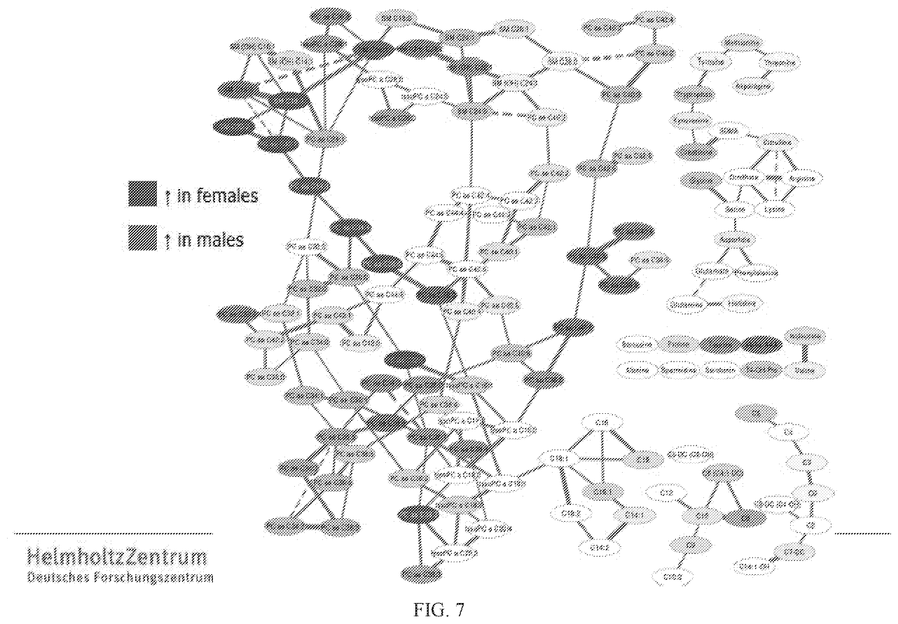

FIGS. 5-7 show the metabolic differences between women and men with disease and highlights additional metabolites like taurine being linked to 2 amino adipic acid.

Results from Imaging Analysis.

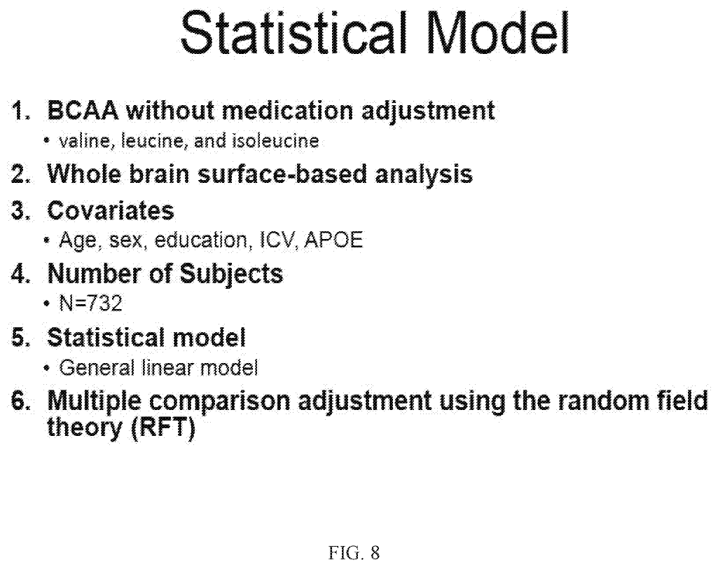

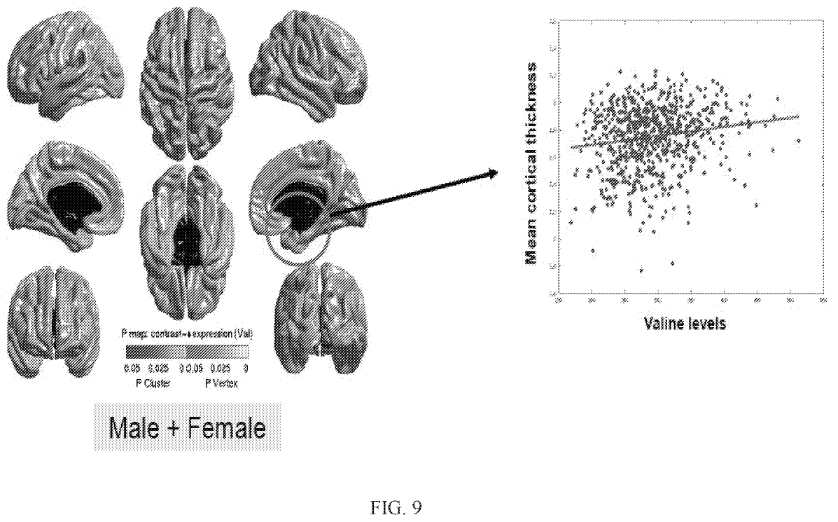

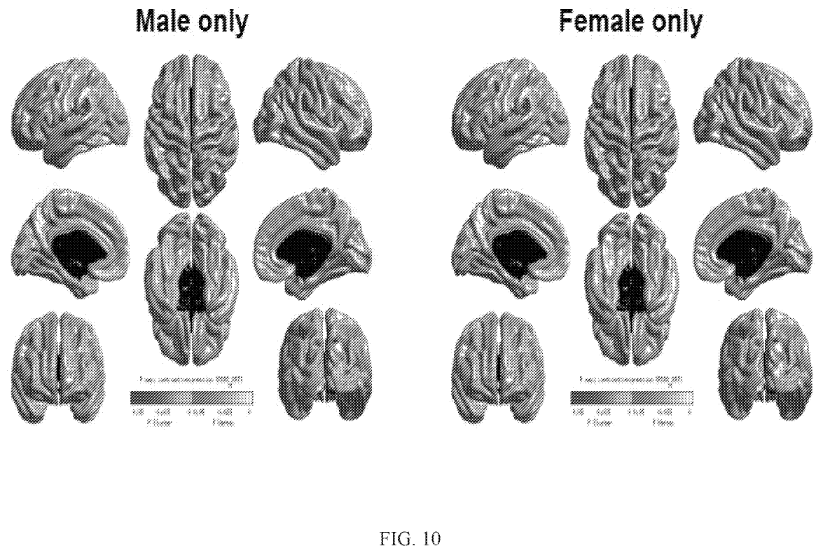

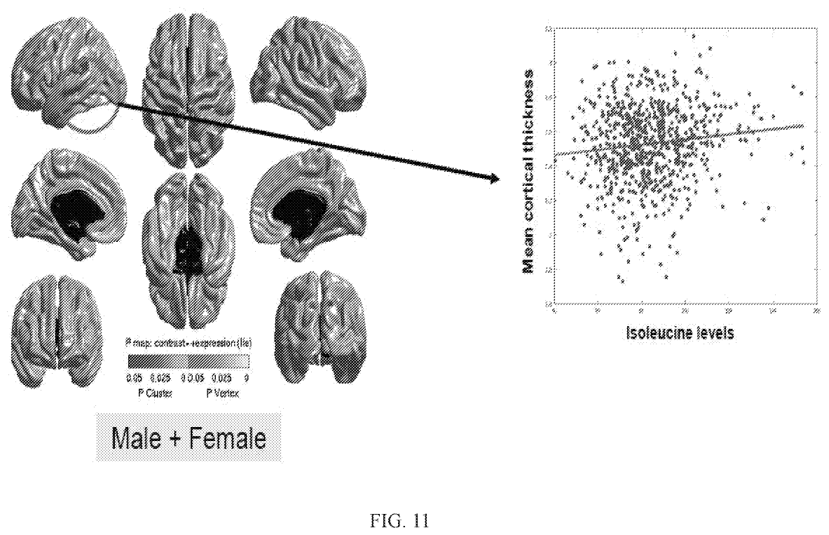

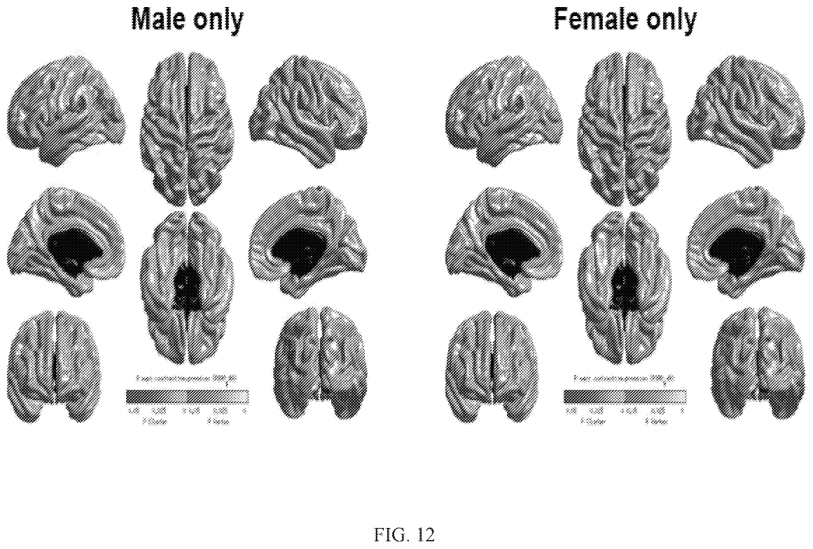

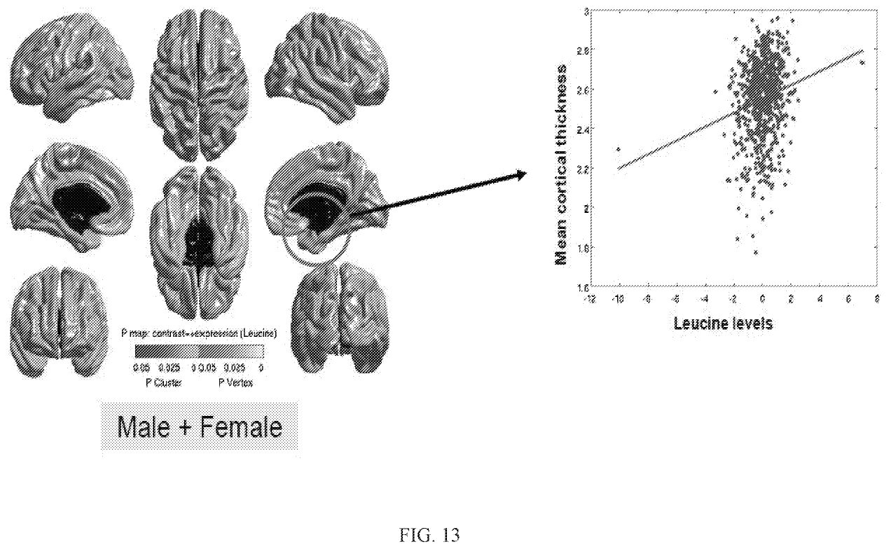

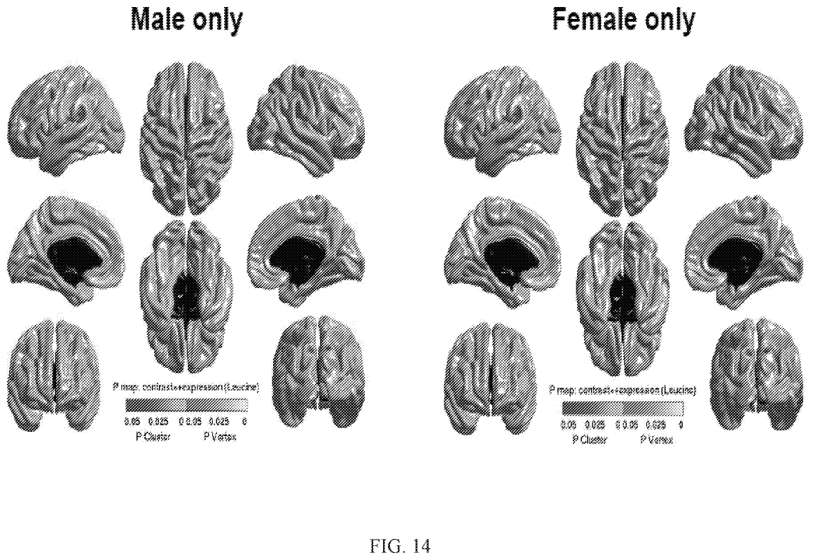

FIG. 8 shows the statistical model used to determine the association of BCAA with imaging phenotypes men and women with Alzheimer disease. Cortical thinning in Alzheimer disease was revealed to be correlated with decreased levels of BCAA in men only. FIGS. 9-14 show the association of BCAA with imaging phenotypes men and women with Alzheimer disease.

Metabolome-Based Genome-Wide Association Study (mGWAS) Analysis.

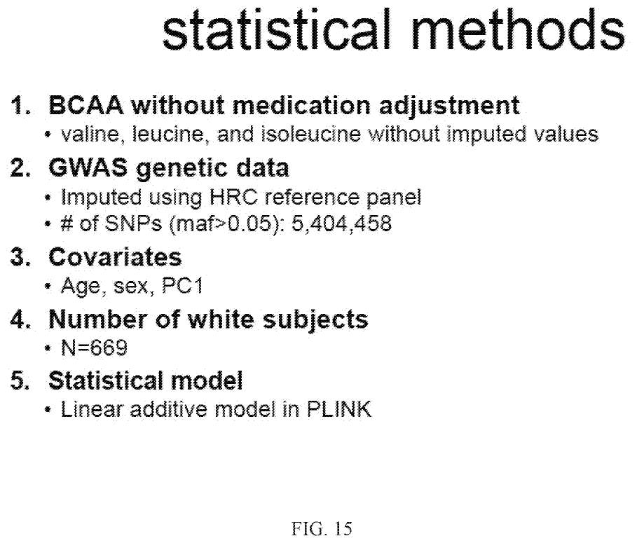

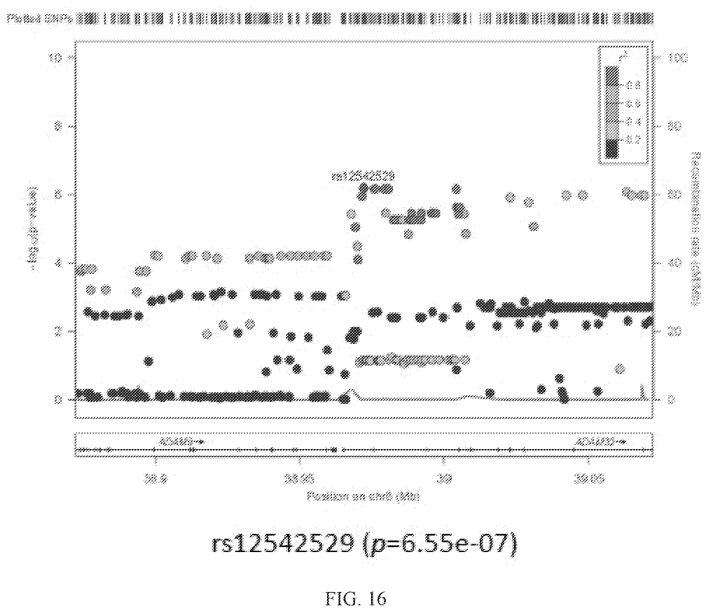

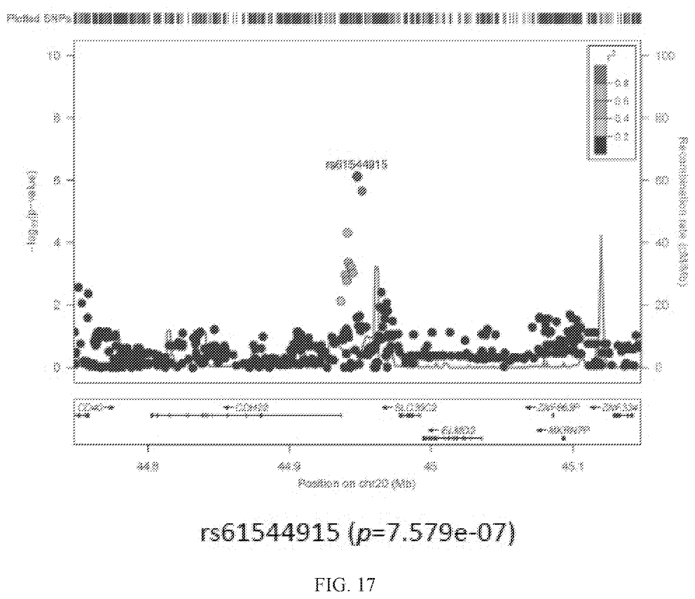

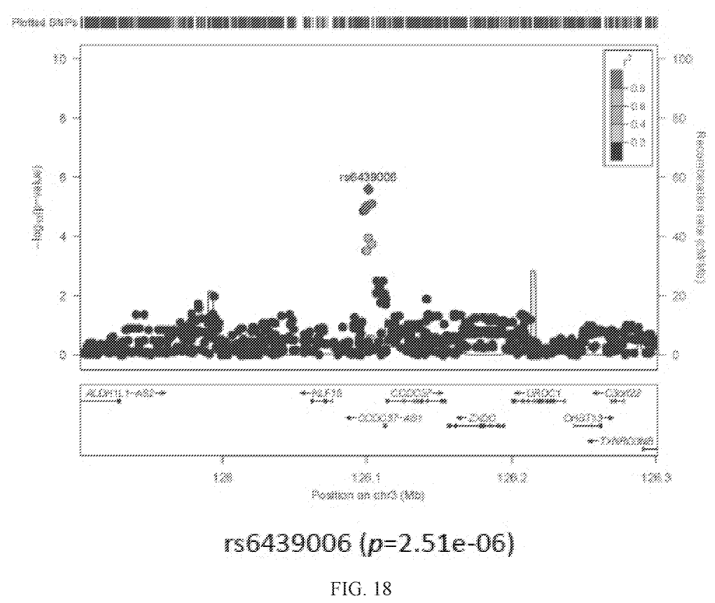

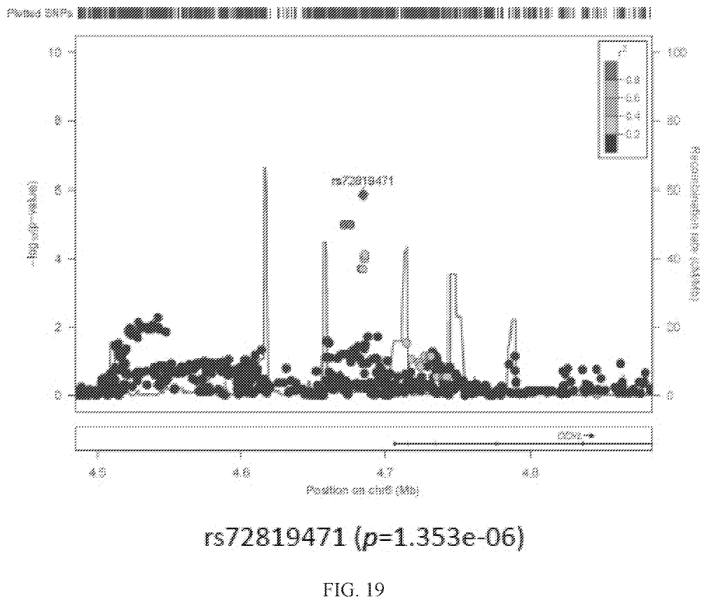

FIG. 15 shows the statistical methods used in the mGWAS analysis. Genetic influences on levels of branched-chain amino acid (BCAA) were investigated by doing mGWAS analysis. A list of putative genes that regulate the levels of these BCAA was identified. They include KLF15, CDYL, CDH22, and ADAM9. FIGS. 16-19 show the association of BCAA with GWAS genetic data.

Whole brain surface-based analysis showed that branched-chain amino acids (valine, leucine, and isoleucine) were significantly associated with cortical thickness atrophy. Higher levels were associated with larger cortical thickness (less cortical thinning) especially in the bilateral temporal lobes including entorhinal cortex. GWAS for branched-chain amino acids (valine, leucine, and isoleucine) identified several marginally associated genes including KLF15, CDYL, CDH22, and ADAM9.

KLF15 orchestrates circadian nitrogen homeostasis. CDYL bridges REST and histone methyltransferases for gene repression and suppression of cellular transformation. CDH22 is expressed predominantly in the brain and the putative calcium-dependent cell adhesion protein may play an important role in morphogenesis and tissue formation in neural and non-neural cells during development and maintenance of the brain and neuroendocrine organs. ADAM9 has a secretase activity and can metabolize Alzheimer's amyloid precursor protein (AAP) towards the nonamyloidogenic pathway. This potentially protects the brain from plaque formation during Alzheimer's disease. Polymorphisms within the ADAM9 promoter region are associated with protection against sporadic Alzheimer's disease.

Key metabolic drivers of AD were identified for each sex. These drivers suggest targets that can be chosen for experimental and functional validation. The findings indicate that different metabolic pathways contributed to AD pathology in males and females and thus therapeutic targets should also be gender-specific. Low levels of BCAA are key drivers of disease in men and should be corrected for in a gender specific manner.

### Example 3

### Branched-Chain Amino Acid Levels Linked with REST/CDYL Regulation

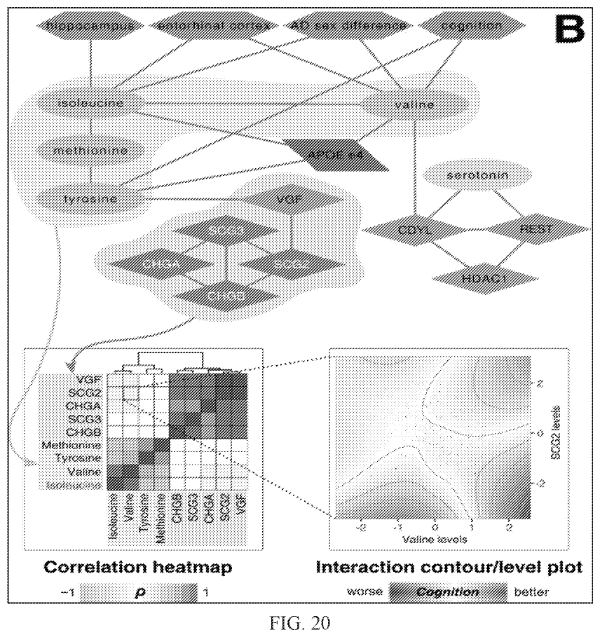

FIG. 20 shows the sub-networks of the target network linking key targets and metabolites. Via metabolic studies, amino acids (AAs) were identified to influence markers of AD. Genetic associations revealed links to the CDYL/REST complex as well as VGF. While mQTLs within CDYL and REST can be explained by their regulation of metabolic enzymes, the link between VGF and AAs was unexpected. VGF and other secretory proteins in the CSF, were negatively correlated with AA levels (heatmap of FIG. 20). Interaction analysis of AA levels and CSF granin/VGF levels revealed significant interaction effects on cognition, exemplified in a contour/level plot for SCG2× valine effects. Interaction models were adjusted for all significant covariates, i.e. age, sex, education, diagnosis, and number of copies of the APOE E4 allele.

The studies in ADNI identified BCAAs to be significantly associated with AD biomarkers, including volumes of hippocampus and the entorhinal cortex, as well as cognitive decline. Among the top results of genetic association analysis with valine levels were variants in the CDYL locus. CDYL is a co-repressor of REST, which has been linked to AD. There may be a shift of REST binding specificity to different gene targets, BCAA degradation pathways on the one hand, neurosecretory pathways on the other, with cognition being impacted if there is an imbalance towards one of the two pathways.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

For reasons of completeness, various aspects of the disclosure are set out in the following numbered clauses:

Clause 1. A method of diagnosing or detecting Alzheimer's disease in a subject, the method comprising: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; wherein if the subject has a level of the at least one biomarker metabolite that is lower that the level of the at least one biomarker metabolite in a control sample, the subject is diagnosed with having Alzheimer's disease.

Clause 2. A method of determining the progression of Alzheimer's disease in a subject, the method comprising: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; wherein if the subject has a level of the at least one biomarker metabolite that is lower that the level of the at least one biomarker metabolite in a control sample, the Alzheimer's disease is determined to be progressing.

Clause 3. A method of determining an increased risk of developing Alzheimer's disease in a subject, the method comprising: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; wherein if the subject has a level of the at least one biomarker metabolite that is lower that the level of the at least one biomarker metabolite in a control sample, the subject is determined to have an increased risk of developing Alzheimer's disease.

Clause 4. A method of aiding in the determination of whether to perform a head magnetic resonance imaging (MRI) procedure on a subject suspected on having Alzheimer's disease, the method comprising: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; c) determining that the subject has an increased risk of cortical thinning if the subject has a level of the at least one biomarker metabolite that is lower that the level of the at least one biomarker metabolite in a control sample; and d) performing a head MRI procedure on the subject that is determined to have cortical thinning.

Clause 5. The method of any one of clauses 1-4, wherein the subject is male and the at least one biomarker metabolite is a branched-chain amino acid biomarker metabolite, a glutamate biomarker metabolite, a tryptophan biomarker metabolite, a tyrosine biomarker metabolite, or a 2 amino adipic acid biomarker metabolite.

Clause 6. The method of clause 5, wherein the branched-chain amino acid biomarker metabolite is valine, leucine, or isoleucine.

Clause 7. The method of any one of clauses 1-4, wherein the subject is female and the at least one biomarker metabolite is a phosphatidylcholine biomarker metabolite, a lysophosphatidylcholine (lysoPC) biomarker metabolite.

Clause 8. The method of any of clauses 1-7, wherein the phosphatidylcholine biomarker metabolite is at least one of Phosphatidylcholine diacyl C36:5 (PC aa C36:5), Phosphatidylcholine diacyl C36:6 (PC as C36:6), Phosphatidylcholine acyl-alkyl C38:0 (PC ae C38:0), Phosphatidylcholine diacyl C38:6 (PC aa C38:6), Phosphatidylcholine acyl-alkyl C40:1 (PC ae C40:1), Phosphatidylcholine diacyl C40:6 (PC aa C40:6), or combinations thereof.

Clause 9. The method of any of clauses 1-8, wherein the lysoPC biomarker metabolite is at least one of lysophosphatidylcholine a C18:2 (lysoPC a C18:2), lysophosphatidylcholine a C18:1 (lysoPC a C18:1), or combinations thereof.

Clause 10. The method of any one of clauses 1-9, wherein the sample from the subject is whole blood, serum, plasma, or cerebral spinal fluid (CSF).

Clause 11. The method of any one of clauses 1-10, wherein the control sample is taken from a subject or population of subjects with normal cognition.

Clause 12. The method of any of clauses 1-11, further comprising determining whether the subject has at least one independent indicator of Alzheimer's disease, wherein the at least one independent indicator of Alzheimer's disease comprises at least one of an increase in Alzheimer's disease Assessment Scale cognitive subscale 13 (ADAS-Cog 13) score, an increase in Spatial Pattern of Abnormality for Recognition of Early Alzheimer's disease (SPARE-AD) score, an increase in brain ventricular volume, presence of Amyloid β 1-42 protein fragment (Aβ1-42), an increased total Tau (T-tau)/Aβ1-42 ratio, or combinations thereof.

Clause 13. The method of any of clauses 1-12, further comprising initiating treatment for Alzheimer's disease in the subject diagnosed with Alzheimer's disease or determined to have an increased risk of developing Alzheimer's disease.

Clause 14. The method of clause 13, wherein if the subject is male and diagnosed with Alzheimer's disease or determined to have an increased risk of developing Alzheimer's disease, the treatment comprises administering a drug that modulates KLF15, CDYL, CDH22, SLC35C2, ADAM32, and/or ADAM9 activity.

Clause 15. The method of clause 14, wherein if the at least one metabolic biomarker is valine, the drug modulates the activity of CDYL, KLF15 and/or CCDC37.

Clause 16. The method of clause 14, wherein if the at least one metabolic biomarker is leucine, the drug modulates the activity of CDH22 and/or SLC35C2.

Clause 17. The method of clause 14, wherein if the at least one metabolic biomarker is isoleucine, the drug modulates the activity of ADAM9 and/or ADAM32.

Clause 18. A method of predicting the outcome of a subject suspected of having Alzheimer's disease, the method comprising: a) obtaining a sample from a subject; b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; wherein if the subject has a level of the at least one biomarker metabolite that is lower that the level of the at least one biomarker metabolite in a control sample, the subject is predicted to develop Alzheimer's disease or have an increased risk of Alzheimer's disease.

Clause 19. The method of clause 18, further comprising initiating treatment for Alzheimer's disease in the subject predicted to develop Alzheimer's disease.

Clause 20. The method of clause 19, wherein if the subject is male and diagnosed with Alzheimer's disease or determined to have an increased risk of developing Alzheimer's disease, the treatment comprises administering a drug that modulates KLF15, CDYL, CDH22, SLC35C2, ADAM32, and/or ADAM9 activity.

Clause 21. A method of treating a subject suspected of having Alzheimer's disease, the method comprising: a) obtaining a sample from a subject; b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof, wherein if the subject has a level of the at least one biomarker metabolite that is lower that the level of the at least one biomarker metabolite in a control sample, the subject is predicted to develop Alzheimer's disease or have an increased risk of Alzheimer's disease, and c) initiating treatment for Alzheimer's disease in the subject predicted to develop Alzheimer's disease.

Clause 22. The method of clause 20 or 21, wherein if the at least one metabolic biomarker is valine, the drug modulates the activity of CDYL, KLF15 and/or CCDC37.

Clause 23. The method of clause 20 or 21, wherein if the at least one metabolic biomarker is leucine, the drug modulates the activity of CDH22 and/or SLC35C2.

Clause 24. The method of clause 20 or 21, wherein if the at least one metabolic biomarker is isoleucine, the drug modulates the activity of ADAM9 and/or ADAM32.

Clause 25. A method of diagnosing or detecting a cognitive disorder in a subject, the method comprising: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; wherein if the subject has a level of the at least one biomarker metabolite that is lower that the level of the at least one biomarker metabolite in a control sample, the subject is diagnosed with having a cognitive disorder.

Clause 26. A method of aiding in the determination of whether to perform a head magnetic resonance imaging (MRI) procedure on a subject suspected on having a cognitive disorder, the method comprising: a) obtaining a sample from a subject; and b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; c) determining that the subject has an increased risk of cortical thinning if the subject has a level of the at least one biomarker metabolite that is lower that the level of the at least one biomarker metabolite in a control sample; and d) performing a head MRI procedure on the subject that is determined to have cortical thinning.

Clause 27. The method of clause 25 or 26, wherein the cognitive disorder is Alzheimer's disease.

Clause 28. A method of predicting the outcome of a subject suspected of having Alzheimer's disease, the method comprising: a) obtaining a sample from a subject; b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof; wherein if the subject has a level of the at least one biomarker metabolite that is lower that the level of the at least one biomarker metabolite in a control sample, the subject is predicted to develop Alzheimer's disease, or an increased risk of Alzheimer's disease.

Clause 29. The method of any one of clauses 25-28, wherein the subject is male and the at least one biomarker metabolite is a branched-chain amino acid biomarker metabolite, a glutamate biomarker metabolite, a tryptophan biomarker metabolite, a tyrosine biomarker metabolite, or a 2 amino adipic acid biomarker metabolite.

Clause 30. The method of any one of clauses 25-28, wherein the subject is female and the at least one biomarker metabolite is a phosphatidylcholine biomarker metabolite, a lysophosphatidylcholine (lysoPC) biomarker metabolite.

Clause 31. The method of any one of clauses 25-30, wherein the branched-chain amino acid biomarker metabolite is valine, leucine, or isoleucine.

Clause 32. The method of any of clauses 25-31, wherein the phosphatidylcholine biomarker metabolite is at least one of Phosphatidylcholine diacyl C36:5 (PC aa C36:5), Phosphatidylcholine diacyl C36:6 (PC aa C36:6), Phosphatidylcholine acyl-alkyl C38:0 (PC ae C38:0), Phosphatidylcholine diacyl C38:6 (PC as C38:6), Phosphatidylcholine acyl-alkyl C40:1 (PC ae C40:1), Phosphatidylcholine diacyl C40:6 (PC as C40:6), or combinations thereof.

Clause 33. The method of any of clauses 25-32, wherein the lysoPC biomarker metabolite is at least one of lysophosphatidylcholine a C18:2 (lysoPC a C18:2), lysophosphatidylcholine a C18:1 (lysoPC a C18:1), or combinations thereof.

Clause 34. The method of any of clauses 25-33, further comprising determining whether the subject has at least one independent indicator of Alzheimer's disease, wherein the at least one independent indicator of Alzheimer's disease comprises at least one of an increase in Alzheimer's disease Assessment Scale cognitive subscale 13 (ADAS-Cog 13) score, an increase in Spatial Pattern of Abnormality for Recognition of Early Alzheimer's disease (SPARE-AD) score, an increase in brain ventricular volume, presence of Amyloid β 1-42 protein fragment (Aβ1-42), an increased total Tau (T-tau)/Aβ1-42 ratio, or combinations thereof.

Clause 35. The method of any of clauses 27-34, further comprising initiating treatment for Alzheimer's disease in the subject that has a level of the at least one biomarker metabolite that is lower that the level of the at least one biomarker metabolite in a control sample.

Clause 36. The method of clause 35, wherein if the subject is male, the treatment comprises administering a composition that modulates CDYL, KLF15, CDH22, SLC35C2, ADAM32, and/or ADAM9 activity.

Clause 37. The method of clause 36, wherein the composition comprises the branched-chain amino acid biomarker metabolite or a drug that modulates the levels of the branched-chain amino acid biomarker metabolite.

Clause 38. The method of clause 37, wherein the treatment comprises administering a composition that modulates CDYL and the composition comprises valine or a drug that modulates the levels of valine.

Clause 39. The method of any one of clauses 25-38, wherein the sample from the subject is whole blood, serum, plasma, or cerebral spinal fluid (CSF).

Clause 40. The method of any one of clauses 25-39, wherein the control sample is taken from a subject or population of subjects with normal cognition.

What is claimed is:

1. A method of diagnosing or detecting a cognitive disorder in a subject, the method comprising:
a) obtaining a sample from a subject; and
b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof;
wherein if the subject has a level of the at least one biomarker metabolite that is lower that the level of the at least one biomarker metabolite in a control sample, the subject is diagnosed with having a cognitive disorder.

2. A method of aiding in the determination of whether to perform a head magnetic resonance imaging (MRI) procedure on a subject suspected on having a cognitive disorder, the method comprising:
a) obtaining a sample from a subject; and
b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof;
c) determining that the subject has an increased risk of cortical thinning if the subject has a level of the at least one biomarker metabolite that is lower that the level of the at least one biomarker metabolite in a control sample; and
d) performing a head MRI procedure on the subject that is determined to have cortical thinning.

3. The method of claim 1 or 2, wherein the cognitive disorder is Alzheimer's disease.

4. The method of claim 3, wherein the subject is male and the at least one biomarker metabolite is a branched-chain amino acid biomarker metabolite, a glutamate biomarker metabolite, a tryptophan biomarker metabolite, a tyrosine biomarker metabolite, or a 2 amino adipic acid biomarker metabolite.

5. The method of claim 3, wherein the subject is female and the at least one biomarker metabolite is a phosphatidylcholine biomarker metabolite, a lysophosphatidylcholine (lysoPC) biomarker metabolite.

6. The method of claim 3, wherein the branched-chain amino acid biomarker metabolite is valine, leucine, or isoleucine.

7. The method of claim 3, wherein the phosphatidylcholine biomarker metabolite is at least one of Phosphatidylcholine diacyl C36:5 (PC aa C36:5), Phosphatidylcholine diacyl C36:6 (PC as C36:6), Phosphatidylcholine acyl-alkyl C38:0 (PC ae C38:0), Phosphatidylcholine diacyl C38:6 (PC as C38:6), Phosphatidylcholine acyl-alkyl C40:1 (PC ae C40:1), Phosphatidylcholine diacyl C40:6 (PC aa C40:6), or combinations thereof.

8. The method of claim 3, wherein the lysoPC biomarker metabolite is at least one of lysophosphatidylcholine a C18:2 (lysoPC a C18:2), lysophosphatidylcholine a C18:1 (lysoPC a C18:1), or combinations thereof.

9. The method of claim 3, further comprising determining whether the subject has at least one independent indicator of Alzheimer's disease, wherein the at least one independent indicator of Alzheimer's disease comprises at least one of an increase in Alzheimer's disease Assessment Scale cognitive subscale 13 (ADAS-Cog 13) score, an increase in Spatial Pattern of Abnormality for Recognition of Early Alzheimer's disease (SPARE-AD) score, an increase in brain ventricular volume, presence of Amyloid β 1-42 protein fragment (Aβ₁₋₄₂), an increased total Tau (T-tau)/Aβ₁₋₄₂ ratio, or combinations thereof.

10. The method of claim 3, further comprising initiating treatment for Alzheimer's disease in the subject that has a level of the at least one biomarker metabolite that is lower that the level of the at least one biomarker metabolite in a control sample.

11. The method of claim 10, wherein if the subject is male, the treatment comprises administering a composition that modulates CDYL, KLF15, CDH22, SLC35C2, ADAM32, and/or ADAM9 activity.

12. The method of claim 11, wherein the composition comprises the branched-chain amino acid biomarker metabolite or a drug that modulates the levels of the branched-chain amino acid biomarker metabolite.

13. The method of claim 12, wherein the treatment comprises administering a composition that modulates CDYL and the composition comprises valine or a drug that modulates the levels of valine.

14. The method of claim 3, wherein the sample from the subject is whole blood, serum, plasma, or cerebral spinal fluid (CSF).

15. The method of claim 3, wherein the control sample is taken from a subject or population of subjects with normal cognition.

16. A method of predicting the outcome of a subject suspected of having Alzheimer's disease, the method comprising:
a) obtaining a sample from a subject;
b) performing biochemical analysis on the sample to measure or detect a level of at least one biomarker metabolite, wherein the at least one biomarker metabolite is selected from the group consisting of a branched-chain amino acid biomarker metabolite, a glutamate biomarker metabolite, a tryptophan biomarker metabolite, a tyrosine biomarker metabolite, a 2 amino adipic acid biomarker metabolite, a phosphatidylcholine biomarker metabolite, a lysophosphatidylcholine biomarker metabolite, and combinations thereof;
wherein if the subject has a level of the at least one biomarker metabolite that is lower that the level of the at least one biomarker metabolite in a control sample, the subject is predicted to develop Alzheimer's disease, or an increased risk of Alzheimer's disease.

17. The method of claim 16, wherein the subject is male and the at least one biomarker metabolite is a branched-chain amino acid biomarker metabolite, a glutamate biomarker metabolite, a tryptophan biomarker metabolite, a tyrosine biomarker metabolite, or a 2 amino adipic acid biomarker metabolite.

18. The method of claim 16, wherein the subject is female and the at least one biomarker metabolite is a phosphatidylcholine biomarker metabolite, a lysophosphatidylcholine (lysoPC) biomarker metabolite.

19. The method of claim 16, wherein the branched-chain amino acid biomarker metabolite is valine, leucine, or isoleucine.

20. The method of claim 16, wherein the phosphatidylcholine biomarker metabolite is at least one of Phosphatidylcholine diacyl C36:5 (PC aa C36:5), Phosphatidylcholine diacyl C36:6 (PC as C36:6), Phosphatidylcholine acyl-alkyl C38:0 (PC ae C38:0), Phosphatidylcholine diacyl C38:6 (PC aa C38:6), Phosphatidylcholine acyl-alkyl C40:1 (PC ae C40:1), Phosphatidylcholine diacyl C40:6 (PC aa C40:6), or combinations thereof.

21. The method of claim 16, wherein the lysoPC biomarker metabolite is at least one of lysophosphatidylcholine a C18:2 (lysoPC a C18:2), lysophosphatidylcholine a C18:1 (lysoPC a C18:1), or combinations thereof.

22. The method of claim 16, further comprising determining whether the subject has at least one independent indicator of Alzheimer's disease, wherein the at least one independent indicator of Alzheimer's disease comprises at least one of an increase in Alzheimer's disease Assessment Scale cognitive subscale 13 (ADAS-Cog 13) score, an increase in Spatial Pattern of Abnormality for Recognition of Early Alzheimer's disease (SPARE-AD) score, an increase in brain ventricular volume, presence of Amyloid β 1-42 protein fragment (Aβ1-42), an increased total Tau (T-tau)/Aβ1-42 ratio, or combinations thereof.

23. The method of claim 16, further comprising initiating treatment for Alzheimer's disease in the subject that has a level of the at least one biomarker metabolite that is lower that the level of the at least one biomarker metabolite in a control sample.

24. The method of claim 23, wherein if the subject is male, the treatment comprises administering a composition that modulates CDYL, KLF15, CDH22, SLC35C2, ADAM32, and/or ADAM9 activity.

25. The method of claim 24, wherein the composition comprises the branched-chain amino acid biomarker metabolite or a drug that modulates the levels of the branched-chain amino acid biomarker metabolite.

26. The method of claim 25, wherein the treatment comprises administering a composition that modulates CDYL and the composition comprises valine or a drug that modulates the levels of valine.

27. The method of claim 16, wherein the sample from the subject is whole blood, serum, plasma, or cerebral spinal fluid (CSF).

28. The method of claim 16, wherein the control sample is taken from a subject or population of subjects with normal cognition.
